SEQUENCE LISTING (63 sequences)

SEQ ID NO: 1 - DNA, 1215 bp, Artificial Sequence
  synthetic
acatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacttgacatccagatgacccagtccccctcctccctgtccgcctccgtgggcgaccgagtaaccatcacctgcaagtccaccaagtccctgctgaactccgacggcttcacctacctggactggtatcagcagaagcctggcaaggcccctaagctgctgatctacctggtgtccgacaggttctccggcgtgccttcccggttcagcggctccggctccggcaccgacttcaccctgaccatctcctccctccagcctgaggacttcgccacctactactgctaagctagctaattaatttaatttggcggcggaacaaaggtggagctcaaacgtggtgggggaggctctggcggcggtggatcgggcggtggtggatctggaggaggtggctcgagcgaggtgcaattggtggagtctggcggcggactggtgcagcctggcggctccctgcggctgtcctgcgccgcctccggctacaccttcaccacctactggatgcactgggtgcggcaggcccctggcaagggcctggagtgggtcgcctacatcaacccttccaccggctataccgagtacaaccagaagttcaaggacaggttcaccatctcccgggacaacgccaagaactccctgtacctccagatgaactccctgcgggccgaggacaccgccgtgtactactgcgccagataagctagctaattaatttaatggggccagggcaccctggtgaccgtgtcctctgatcaggcctcaggggcc

SEQ ID NO: 2 - DNA, 1909 bp, Artificial
  synthetic
ggtaccacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacagtccgacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagctcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaataaggcgcgccaattctatttcaaggagacagtcataatgaaatacctgctgccgaccgctgctgctggtctgctgctcctcgcggcccagccggcaatggcctcgagcgaggtgcaattggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtggggcctcaggggccattaattaa

SEQ ID NO: 3 - protein, 16 aa, Artificial
  synthetic
DGGGSGGGGSGGGGSS

SEQ ID NO: 4 - DNA, 11 bp, Artificial
  misc_feature (4)..(8): synthetic; n is a, c, g, or t
ccannnnntgg

SEQ ID NO: 5 - DNA, 10 bp, Artificial
  misc_feature (3)..(7): synthetic; n is a, c, g, or t
gcnnnnnggc

SEQ ID NO: 6 - DNA, 10 bp, Artificial
  misc_feature (4)..(7): synthetic; n is a, c, g, or t
gatnnnnatc

SEQ ID NO: 7 - DNA, 12 bp, Artificial
  misc_feature (4)..(9): synthetic; n is a, c, g, or t
ccannnnnntgg

SEQ ID NO: 8 - DNA, 11 bp, Artificial
  misc_feature (4)..(8): synthetic; n is a, c, g, or t
gacnnnnngtc

SEQ ID NO: 9 - DNA, 15 bp, Artificial
  synthetic
ctctcttaaggtagc

SEQ ID NO: 10 - DNA, 13 bp, Artificial
  misc_feature (5)..(9): synthetic; n is a, c, g, or t
ggccnnnnnggcc

SEQ ID NO: 11 - DNA, 10 bp, Artificial
  misc_feature (4)..(7): synthetic; n is a, c, g, or t
gaannnnttc

SEQ ID NO: 12 - DNA, 13 bp, Artificial
  synthetic
gccgccaccatgg

SEQ ID NO: 13 - DNA, 48 bp, Artificial
  synthetic
gatggcggtggatcgggcggtggtggatctggaggaggtggctcgagc

SEQ ID NO: 14 - protein, 7 aa, Homo sapiens
TLVTVSS

SEQ ID NO: 15 - DNA, 21 bp, Homo sapiens
accctggtcaccgtctcctac

SEQ ID NO: 16 - protein, 5 aa, Artificial
  synthetic
SHSAQ

SEQ ID NO: 17 - DNA, 15 bp, Artificial
  synthetic
tctcacagtgcacag

SEQ ID NO: 18 - protein, 7 aa, Homo sapiens
TKVEIKR

SEQ ID NO: 19 - protein, 7 aa, Artificial
  synthetic
TKVELKR

SEQ ID NO: 20 - DNA, 21 bp, Artificial
  synthetic
accaagctggagctcaaacgt

SEQ ID NO: 21 - DNA, 24 bp, Artificial
  synthetic
tggaagaggcacgttcttttcttt

SEQ ID NO: 22 - DNA, 24 bp, Artificial
  synthetic
acactctcccctgttgaagctctt

SEQ ID NO: 23 - DNA, 44 bp, Artificial
  synthetic
ggctatggttgcggctcgagccagrtgcagctggtgcartctgg

SEQ ID NO: 24 - DNA, 44 bp, Artificial
  synthetic
ggctatggttgcggctcgagcsaggtccagctggtrcagtctgg

SEQ ID NO: 25 - DNA, 44 bp, Artificial
  synthetic
ggctatggttgcggctcgagccagrtcaccttgaaggagtctgg

SEQ ID NO: 26 - DNA, 44 bp, Artificial
  synthetic
ggctatggttgcggctcgagcsaggtgcagctggtggagtctgg

SEQ ID NO: 27 - DNA, 44 bp, Artificial
  synthetic
ggctatggttgcggctcgagcgaggtgcagctggtggagwcygg

SEQ ID NO: 28 - DNA, 44 bp, Artificial
  synthetic
ggctatggttgcggctcgagccaggtgcagctacagcagtgggg

SEQ ID NO: 29 - DNA, 44 bp, Artificial
  synthetic
ggctatggttgcggctcgagccagstgcagctgcaggagtcsgg

SEQ ID NO: 30 - DNA, 44 bp, Artificial
  synthetic
ggctatggttgcggctcgagcgargtgcagctggtgcagtctgg

SEQ ID NO: 31 - DNA, 44 bp, Artificial
  synthetic
ggctatggttgcggctcgagccaggtacagctgcagcagtcagg

SEQ ID NO: 32 - DNA, 44 bp, Artificial
  synthetic
ggctatggttgcagtgcacttgacatccagwtgacccagtctcc

SEQ ID NO: 33 - DNA, 44 bp, Artificial
  synthetic
ggctatggttgcagtgcacttgatgttgtgatgactcagtctcc

SEQ ID NO: 34 - DNA, 44 bp, Artificial
  synthetic
ggctatggttgcagtgcacttgaaattgtgwtgacrcagtctcc

SEQ ID NO: 35 - DNA, 44 bp, Artificial
  synthetic
ggctatggttgcagtgcacttgatattgtgatgacccacactcc

SEQ ID NO: 36 - DNA, 44 bp, Artificial
  synthetic
ggctatggttgcagtgcacttgaaacgacactcacgcagtctcc

SEQ ID NO: 37 - DNA, 44 bp, Artificial
  synthetic
ggctatggttgcagtgcacttgaaattgtgctgactcagtctcc

SEQ ID NO: 38 - DNA, 55 bp, Artificial
  synthetic
ggctatggttgcggcccctgaggcctgatcatgaggagacggtgaccagggtgcc

SEQ ID NO: 39 - DNA, 55 bp, Artificial
  synthetic
ggctatggttgcggcccctgaggcctgatcatgaagagacggtgaccattgtccc

SEQ ID NO: 40 - DNA, 55 bp, Artificial
  synthetic
ggctatggttgcggcccctgaggcctgatcatgaggagacggtgaccagggttcc

SEQ ID NO: 41 - DNA, 55 bp, Artificial
  synthetic
ggctatggttgcggcccctgaggcctgatcatgaggagacggtgaccgtggtccc

SEQ ID NO: 42 - DNA, 36 bp, Artificial
  synthetic
ggctatggttgcacgtttgagctccaccttggtccc

SEQ ID NO: 43 - DNA, 36 bp, Artificial
  synthetic
ggctatggttgcacgtttgagctccagcttggtccc

SEQ ID NO: 44 - DNA, 36 bp, Artificial
  synthetic
ggctatggttgcacgtttgagctccactttggtccc

SEQ ID NO: 45 - DNA, 36 bp, Artificial
  synthetic
ggctatggttgcacgtttgagctccagtcgtgtccc

SEQ ID NO: 46 - DNA, 71 bp, Artificial
  synthetic
gatggcggtggatcgggcggtggtggatctggaggaggtggctcgagccagrtgcagctggtgcartctgg

SEQ ID NO: 47 - DNA, 71 bp, Artificial
  synthetic construct
gatggcggtggatcgggcggtggtggatctggaggaggtggctcgagcsaggtccagctggtrcagtctgg

SEQ ID NO: 48 - DNA, 71 bp, Artificial
  synthetic construct
gatggcggtggatcgggcggtggtggatctggaggaggtggctcgagccagrtcaccttgaaggagtctgg

SEQ ID NO: 49 - DNA, 71 bp, Artificial
  synthetic construct
gatggcggtggatcgggcggtggtggatctggaggaggtggctcgagcsaggtgcagctggtggagtctgg

SEQ ID NO: 50 - DNA, 71 bp, Artificial
  synthetic construct
gatggcggtggatcgggcggtggtggatctggaggaggtggctcgagcgaggtgcagctggtggagwcygg

SEQ ID NO: 51 - DNA, 71 bp, Artificial
  synthetic construct
gatggcggtggatcgggcggtggtggatctggaggaggtggctcgagccaggtgcagctacagcagtgggg

SEQ ID NO: 52 - DNA, 71 bp, Artificial
  synthetic construct
gatggcggtggatcgggcggtggtggatctggaggaggtggctcgagccagstgcagctgcaggagtcsgg

SEQ ID NO: 53 - DNA, 71 bp, Artificial
  synthetic construct
gatggcggtggatcgggcggtggtggatctggaggaggtggctcgagcgargtgcagctggtgcagtctgg

SEQ ID NO: 54 - DNA, 71 bp, Artificial
  synthetic construct
gatggcggtggatcgggcggtggtggatctggaggaggtggctcgagccaggtacagctgcagcagtcagg

SEQ ID NO: 55 - DNA, 72 bp, Artificial
  synthetic construct
gctcgagccacctcctccagatccaccaccgcccgatccaccgccatcacgtttgagctccaccttggtccc

SEQ ID NO: 56 - DNA, 72 bp, Artificial
  synthetic construct
gctcgagccacctcctccagatccaccaccgcccgatccaccgccatcacgtttgagctccagcttggtccc

SEQ ID NO: 57 - DNA, 72 bp, Artificial
  synthetic construct
gctcgagccacctcctccagatccaccaccgcccgatccaccgccatcacgtttgagctccactttggtccc

SEQ ID NO: 58 - DNA, 72 bp, Artificial; identical to SEQ ID NO: 55
  synthetic construct

SEQ ID NO: 59 - DNA, 72 bp, Artificial
  synthetic construct
gctcgagccacctcctccagatccaccaccgcccgatccaccgccatcacgtttgagctccactcgtgtccc

SEQ ID NO: 60 - DNA, 4513 bp, Artificial
  synthetic construct
ccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgggtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgagcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattcaggaggaatttaaaatgaaaaagacagctatcgcgattgcagtggcactggctggtttcgctaccgtggcccaggcggcccccctatttgtttatttttctaaatacattcaaatatgtatccgctcatgagacaataaccctgataaatgcttcaataatgtgaggagggccaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaatggccaagtgatgattgaccagtgccgttccggtgctcaccgcgtgagacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaggcctcaggggcccaccatcaccatcaccatggcgcagaattcgagcagaagctgatctctgaggaagacctgtagggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaattaatttaagcggccgcagatctgggaaattgtaagcgttaatat

SEQ ID NO: 61 - DNA, 562 bp, Artificial
  synthetic construct
acatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacgagctcaaacgtggtgggggaggctctggcggcggtggatcgggcggtggtggatctggaggaggtggctcgagcctggtgaccgtgtcctctgatcaggcctcaggggcc

SEQ ID NO: 62 - DNA, 4178 bp, Artificial
  synthetic construct
ccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgggtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgagcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacaggcgcgccacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctattctcacagtgcacttgacatccagatgacccagtccccctcctccctgtccgcctccgtgggcgaccgagtaaccatcacctgcaagtccaccaagtccctgctgaactccgacggcttcacctacctggactggtatcagcagaagcctggcaaggcccctaagctgctgatctacctggtgtccgacaggttctccggcgtgccttcccggttcagcggctccggctccggcaccgacttcaccctgaccatctcctccctccagcctgaggacttcgccacctactactgctaagctagctaattaatttaatttggcggcggaacaaaggtggagctcaaacgtggtgggggaggctctggcggcggtggatcgggcggtggtggatctggaggaggtggctcgagcgaggtgcaattggtggagtctggcggcggactggtgcagcctggcggctccctgcggctgtcctgcgccgcctccggctacaccttcaccacctactggatgcactgggtgcggcaggcccctggcaagggcctggagtgggtcgcctacatcaacccttccaccggctataccgagtacaaccagaagttcaaggacaggttcaccatctcccgggacaacgccaagaactccctgtacctccagatgaactccctgcgggccgaggacaccgccgtgtactactgcgccagataagctagctaattaatttaatggggccagggcaccctggtgaccgtgtcctctgatcaggcctcaggggcccaccatcaccatcaccatggcgcagaattcgagcagaagctgatctctgaggaagacctgtagggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaattaatttaagcggccgcagatctgggaaattgtaagcgttaatat

SEQ ID NO: 63 - DNA, 4849 bp, Artificial
  synthetic construct
ccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgggtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgagcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattcaggaggaatttaaaatgaccatgattacgccaagctttggagccttttttttggagattttcaacgtgaaaaaattattattcgcaattcctttagttgttcctttctattctcacagtgcacagtccgacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagctcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaataaggcgcgccaattctatttcaaggagacagtcataatgaaatacctgctgccgaccgctgctgctggtctgctgctcctcgcggcccagccggcaatggcctcgagcgaggtgcaattggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtggggcctcaggggcccaccatcaccatcaccatggcgcagaattcgagcagaagctgatctctgaggaagacctgtagggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaattaatttaagcggccgcagatctgggaaattgtaagcgttaatat

(19) United States
(12) Patent Application Publication
(10) Pub. No.: US 2010/0124764 A1
(43) Pub. Date: May 20, 2010

(54) SINGLE CHAIN ANTIBODY LIBRARY DESIGN

(75) Inventors: Simon Evan Hufton, Hitchin (GB); William James Jonathan Finlay, Dublin (IE); Ian David Broadbent, Cults (GB); Laird Bloom, Needham, MA (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/566,767

(22) Filed: Sep. 25, 2009

Related U.S. Application Data
(60) Provisional application No. 61/100,350, filed on Sep. 26, 2008.

(51) Int. Cl. C12P 21/00; C07H 21/04; C40B 50/00; C40B 40/06

(52) U.S. Cl. 435/69.1; 536/23.1; 506/23; 506/16

(57) ABSTRACT

The invention provides polynucleotide vectors and linkers and methods for designing and making single chain variable fragment (“ScFv”) libraries. The invention also provides polynucleotide vectors and linkers and methods for reformatting the ScFv library into Fab and IgG formats for high throughput production and screening.

This application claims priority to U.S. Provisional Application No. 61/100,350, filed on Sep. 26, 2008 which is incorporated herein by reference in its entirety.

### BACKGROUND

The present invention relates to single chain Fv (ScFv) libraries, and vectors and methods for screening and reformatting those libraries.

Protein therapeutics are an important part of drug discovery. High-throughput screening of large libraries of protein variants allows efficient discovery or optimization of protein therapeutics for desirable properties such as binding affinity, stability and specificity.

Therapeutic antibodies are particularly attractive because of their high affinity and specificity to the antigen and because of their relatively high stability in vitro and in vivo. Antibodies are made of two heavy and two light chains, which contain the variable regions at their N-termini and which are linked by disulfide bridges. Single-chain antibodies have been engineered by linking fragments of the variable heavy and light chain regions (ScFv).

Typical procedures for making ScFv generally involve amplification of gene regions encoding the variable regions of the antibodies, assembly of an ScFv genetic sequence and expression of the ScFv genetic sequence in host cells. The host cells are screened using a target antigen to identify those cells which bind to the antigen, and thus which express a functional ScFv of the desired specificity.

The most commonly used techniques to identify single-chain antibodies which bind specific antigens is by phage display and variations thereof (see Hoogenboom et al., 1998). Generally, phage display methods involve the insertion of random oligonucleotides into a phage genome such that they direct a bacterial host to express peptide libraries fused to phage coat proteins (e.g., filamentous phage pIII, pVI or pVIII). Libraries of up to 10¹⁰ individual members can be routinely prepared in this way. Incorporation of the ScFv sequences into the mature phage coat sequence results in the ScFv antibodies encoded by the heterologous sequence being displayed on the exterior surface of the phage. By immobilizing a relevant antigen target (or targets) to a surface, a phage that displays an ScFv that binds to one of those targets on the surface will remain while others are removed by washing.

However, ScFv can have a tendency to dimerise which can complicate screening assays particularly when quantitative discrimination between selected clones is desired.

### SUMMARY

The present invention provides a single chain antibody (ScFv) library design using generic restriction sites that facilitate “bulk” reformatting of ScFv antibodies into other immunoglobulin formats, e.g., Fabs, IgGs, ScFv-Fc fusions, for protein expression or display. Thus, the present invention allows transfer and screening of larger pools of ScFv in a desirable molecular format at an earlier stage in the drug discovery or optimization process, thereby shortening the drug discovery timeline.

In one aspect, the present invention provides a polynucleotide that serves as a multiple cloning site or linker (a.k.a. hereinafter as polynucleotide linker, polylinker or MCS) comprising at least four restriction enzyme recognition sites to accommodate the cloning (insertion and ligation) of an antibody heavy chain variable region (“VH”) polynucleotide fragment and an antibody light chain variable region (“VL”).

In one embodiment, the sites for cloning the VH polynucleotide are located 5-prime (5′) to the sites for cloning the VL polynucleotide. In this particular embodiment, the polynucleotide linker comprises a first restriction site (“site 1”) located at the 5′ end of the site for the VH polynucleotide, a second restriction site (“site 2”) located at the 3-prime (3′) end of the site for the VH polynucleotide, a third restriction site (“site 3”) located 3′ of site 2 and at the 5′ end of the site for the VL polynucleotide, and a fourth restriction site (“site 4”) located at the 3-prime (3′) end of the site for the VL polynucleotide. In some embodiments, site 1 is selected from the group comprising SfiI, BssHII, ApaLI and MfeI; site 2 is selected from the group comprising XhoI, Sall, BcII, BstEII, Mlul, Smal and XbaI; site 3 is selected from the group consisting of XhoI, Sall, BspEi, ApaLI, BssHII and EcoRV; and site 4 is selected from the group comprising SfiI, BclL, AvrII, BsiWI and BamHl. In some embodiments, site 1 is BssHI; site 2 is XhoI; site 3 is BspEI; and site 4 for is AvrII, when the VL polynucleotide encodes a lambda light-chain polypeptide, and BsiWI, when the VL polynucleotide encodes a kappa light-chain polypeptide.

In another embodiment, the sites for cloning the VL polynucleotide are located 5-prime (5′) to the sites for cloning the VH polynucleotide. In this particular embodiment, the polynucleotide linker comprises a first restriction site (“site 1”) located at the 5′ end of the site for the VL polynucleotide, a second restriction site (“site 2”) located at the 3-prime (3′) end of the site for the VL polynucleotide, a third restriction site (“site 3”) located 3′ of site 2 and at the 5′ end of the site for the VH polynucleotide, and a fourth restriction site (“site 4”) located at the 3-prime (3′) end of the site for the VH polynucleotide. In some embodiments, site 1 is selected from the group comprising SfiI, BssHII and ApaLI; site 2 is selected from the group comprising XhoI, Sall, BcII, SacI, AvrII, BsiWI and Mlul; site 3 is selected from the group consisting of MfeI, BspEI, ApaLI, BssHII, XhoI and Sall; and site 4 is selected from the group comprising SfiI, BcII, XhoI, Sall and BstEII. In some embodiments, site 1 is BssHI; site 2 is AvrII when the VL polynucleotide encodes a lambda light-chain polypeptide, and BsiWI when the VL polynucleotide encodes a kappa light-chain polypeptide; site 3 is BspEI; and site 4 for is XhoI.

In another aspect, the present invention provides a polynucleotide that serves as a multiple cloning site or linker (a.k.a. hereinafter as polynucleotide linker, polylinker or MCS) containing a nucleotide sequence, when double-stranded, forming restriction enzyme recognition sites including, for example, from 5′ to 3′, an ApaLI site, a SacI site, an XhoI site and a SfiI Site. In some embodiments, the polynucleotide linker further includes a BstEII site 3′ to the XhoI site and 5′ to the SfiI site.

In another aspect, the present invention provides a polynucleotide linker containing a polynucleotide sequence, when double-stranded, forming restriction enzyme recognition sites including, for example, from 5′ to 3′, an AscI site, a PciI site, a HindIII site, an ApaLI site, a SacI site, an AvaI site, an XhoI site, an MfeI site, an Xmal site, a Smal site, a BstEII site, a BcII site, and a SfiI site.

In some embodiments, the polynucleotide sequence includes a nucleotide sequence of SEQ ID NO:1

In some embodiments, the polynucleotide sequence of this aspect of the invention includes a nucleotide sequence at least 50%, 55%, 60%, 65%, 70%, 75%, 80%, 85%, 90%, 95%, 96%, 97%, 98%, 99%, or 99.5% identical to SEQ ID NO:1. In certain embodiments, the polynucleotide sequence includes a nucleotide sequence at least 95% identical to SEQ ID NO:1.

In yet another aspect, the present invention provides a polynucleotide linker containing a polynucleotide sequence, when double-stranded, forming restriction enzyme recognition sites including, for example, from 5′ to 3′, a PciI site, a HindIII site, an ApaLI site, a SacI site, an AscI site, an XhoI site, an AvaI site, an MfeI site, a BstEII site, and a SfiI site.

In some embodiments, the polynucleotide sequence contains a nucleotide sequence of SEQ ID NO:2.

In some embodiments, the polynucleotide sequence of this aspect of the invention includes a nucleotide sequence at least 50%, 55%, 60%, 65%, 70%, 75%, 80%, 85%, 90%, 95%, 96%, 97%, 98%, 99%, or 99.5% identical to SEQ ID NO:2. In certain embodiments, the polynucleotide sequence includes a nucleotide sequence at least 95% identical to SEQ ID NO:2.

The present invention further provides vectors containing a polynucleotide linker as described in various embodiments above.

In one aspect, the present invention provides a nucleic acid construct containing a variable heavy chain (VH) gene and a variable light chain (VL) gene. The nucleic acid construct further contains an XhoI restriction site at 5′ end of the VH gene, a SfiI or BstEII restriction site at 3′ end of the VH gene, an ApaL1 restriction site at 5′ end of the VL gene and a Sac1 restriction site at 3′ end of the VL gene.

In some embodiments, the VL gene and the VH gene are in a VL-VH format. In other embodiments, the nucleic acid construct includes a linker between the VH gene and the VL gene. In some embodiments, the linker includes a sequence such as DGGGSGGGGSGGGGSS (SEQ ID NO:3).

In another aspect, the present invention provides a single chain Fv (ScFv) library including a plurality of nucleic acid constructs, each of which contains a VH gene and a VL gene, an XhoI restriction site at 5′ end of the VH gene, a SfiI or BstEII restriction site at 3′ end of the VH gene, an ApaL1 restriction site at 5′ end of the VL gene and a Sac1 restriction site at 3′ end of the VL gene.

In some embodiments, the VL gene and the VH gene are in a VL-VH format in each nucleic acid construct. In some embodiments, each of the plurality of nucleic acid constructs includes a linker between the VH gene and the VL gene. In some embodiments, the linker includes a sequence such as DGGGSGGGGSGGGGSS (SEQ ID NO:3).

In a further aspect, the present invention provides a method for constructing a single chain Fv (ScFv) library. The method includes the steps of: (1) introducing an XhoI restriction site to the 5′ end and a SfiI or BstEII restriction site to the 3′ end of a collection of VH genes; (2) cloning the collection of VH genes into a plurality of vectors using a first restriction site compatible with XhoI and a second restriction site compatible with Sfi1 or BstEII; (3) introducing a SacI restriction site to the 5′ end and an ApaLI restriction site to the 3′ end of a collection of VL genes; and (4) cloning the collection of VL genes into the plurality of vectors using restriction sites compatible with ApaL1 and Sac1.

In some embodiments, methods for constructing a single chain Fv (ScFv) library can include synthesizing first strand cDNA from isolated total RNA and amplifying VH genes by PCR amplification using one or more primer sets including a forward primer containing an XhoI restriction site and a reverse primer containing a SfiI or BstEII restriction site.

In some embodiments, methods for constructing a single chain Fv (ScFv) library can include synthesizing first strand cDNA from isolated total RNA and amplifying VL genes by PCR amplification using one or more primer sets including a forward primer containing an ApaL1 restriction site and a reverse primer containing a Sac1 restriction site.

The present invention further provides a single-chain Fv (ScFv) library constructed using the method of this aspect of the invention.

In yet another aspect, the present invention provides a method for reformatting a single-chain Fv (ScFv) library into an Fab expression system. The method includes the steps of: (1) providing an ScFv library of the present invention; (2) generating a plurality of fragments, each of which includes a VH gene and a VL gene, by digesting the ScFv library using one or more restriction enzymes; and (3) cloning the plurality of fragments generated from step (2) to a plurality of Fab expression vectors with compatible restriction sites.

In some embodiments, the one or more restriction enzymes include ApaLI and BstEII. In some embodiments, the VH gene and the VL gene are in a VL-VH format. In some embodiments, each of the plurality of fragments further includes a linker between the VH and the VL gene. In some embodiments, the method further includes a step of replacing at least a portion of the linker with a sequence containing a Ck sequence, a ribosome binding site (rbs) and a signal peptide sequence. In some embodiments, the signal peptide sequence contains a PelB leader sequence.

In still another aspect, the present invention provides a method for reformatting a single-chain Fv (ScFv) library into an IgG expression system. The method includes the steps of: (1) providing an ScFv library of the present invention; (2) generating a plurality of fragments, each of which comprises a VH gene and a VL gene, by digesting the ScFv library using one or more restriction enzymes; and (3) cloning the plurality of fragments generated from step (2) to a plurality of IgG expression vectors with compatible restriction sites.

In some embodiments, the one or more restriction enzymes include ApaL1 and BstEII. In some embodiments, the VH gene and the VL gene are in a VL-VH format. In some embodiments, each of the plurality of fragments further includes a linker between the VH and the VL gene. In some embodiments, the method further includes a step of replacing at least a portion of the linker with a sequence containing a Ck sequence, an internal ribosome entry site (IRES) and a signal peptide sequence.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

FIG. 1 illustrates exemplary V gene amplification strategy.

FIG. 2 illustrates an exemplary vector containing an ScFv dummy construct.

FIG. 3 illustrates an exemplary restriction-based cloning strategy for constructing an ScFv library.

FIG. 4 illustrates an exemplary two-step procedure batch reformatting an ScFv library to an Fab library for high throughput protein expression. An exemplary polylinker of an Fab vector is also shown.

FIG. 5 illustrates an exemplary two-step procedure batch reformatting an ScFv library to an IgG expression system for high throughput protein expression.

### DETAILED DESCRIPTION

The present invention provides single chain Fv (ScFv) library designs using compatible restriction sites that allow seamless conversion of pools of ScFv polypeptides into different molecular formats, e.g., Fabs, IgGs or Fc-fusions, for both high throughput expression and library display. In particular, the present invention provides vectors and methods for making ScFv libraries based on compatible restriction sites and methods of reformatting the ScFv libraries of the invention into Fabs, IgGs or other immunoglobulin formats using the compatible restriction sites.

Various aspects of the invention are described in detail in the following sections. The use of sections is not meant to limit the invention. Each section can apply to any aspect of the invention.

### Single Chain Variable (ScFv) Polypeptide

A typical single chain variable fragment (ScFv) is a recombinant polypeptide containing the variable regions of the heavy and light chains of immunoglobulins, linked together with a linker. Typically, the linker is short and flexible. In some embodiments, the linker links the carboxyl terminus of the VL to the amino terminus of the VH sequence (VL-VH configuration). In other embodiments, the linker links the carboxyl terminus of the VH to the amino terminus of the VL sequence (VH-VL configuration). In other embodiments, the ScFv polypeptide retains the specificity of the original immunoglobulin. General methods for constructing a DNA encoding an ScFv polypeptide are known. A ScFv polypeptide is encoded by a ScFv polynucleotide.

As used in this application, an ScFv polypeptide is also referred to as a displayed or expressed polypeptide, a polypeptide of interest, or a heterologous polypeptide. In addition, terms “polypeptide,” “peptide,” or “protein” are used interchangeably in this application.

### Vectors

As used herein, the term “vector” or “polynucleotide vector” refers to a nucleic acid molecule capable of carrying and transferring another nucleic acid fragment or sequence to which it has been linked from one location (e.g., a host, a system) to another. The term includes vectors for in vivo or in vitro expression systems, such as e.g. polynucleotide expression vectors. For example, vectors of the invention can be in the form of “plasmids” which refer to circular double stranded DNA loops which are typically maintained episomally. Vectors of the invention can also be in linear forms. In addition, the invention is intended to include other forms of vectors which serve equivalent functions and which become known in the art subsequently hereto.

Vectors of the present invention can be used for the expression of polynucleotides and polypeptides. Generally, the vectors of the invention include cis-acting regulatory regions operably linked to the polynucleotide to be expressed. The regulatory regions may be constitutive or inducible. Appropriate trans-acting factors either are supplied by the host, by the in vitro translation system, by a complementing vector, or by the vector itself upon introduction into the host.

The vectors of the invention can be derived from bacterial plasmids, from bacteriophage, from yeast episomes, from yeast chromosomal elements, from mammalian viruses, from mammalian chromosomes, and from combinations thereof, such as those derived from plasmid and bacteriophage genetic elements including, but not limited to, cosmids and phagemids.

The vectors of the invention can include any elements that typically included in an expression or display vector including, but not limited to, origin or replication sequences, anti-biotic resistance genes, leader or signal peptide sequences, various tag sequences, stuffer sequences, and restriction sites.

General methods for constructing vectors of the present invention are well known in the art. For example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

The present invention also provides host cells or other organisms that contain the vectors of the invention. For example, the present invention provides bacteria, mammalian cells, yeast and other cellular system containing the vectors of the invention. Suitable mammalian cells include, but are not limited to, Chinese hamster ovary cells, HeLa cells, baby hamster kidney cells, NSO mouse melanoma cells and many others. An exemplary common bacterial host is E. coli.

### Polynucleotide Linker

Typically, a vector of the invention includes a polynucleotide linker containing one or more compatible restriction sites that facilitate transfer of antibody heavy chain variable (VH) and light chain variable (VL) genes between libraries using restriction site-based cloning strategy. As used herein, the term “polynucleotide linker” or “polylinker” refers to a polynucleotide sequence containing at least one restriction enzyme recognition site, or a single-stranded polynucleotide sequence, when double-stranded, forming at least one restriction enzyme recognition site.

As used herein, the term “compatible restriction site” refers to a restriction site on a vector of one format (e.g., ScFv phage display vector) that is compatible with at least a restriction site on a vector with different molecular format (e.g., Fab or IgG format). As used herein, restriction sites are “compatible” if, once cleaved by appropriate restriction enzymes, can be ligated by a DNA ligase. In some embodiments, the compatible restriction sites include those double-stranded sequences that, once cleaved by appropriate restriction enzymes, generate “sticky ends” with complementary overhang sequences that can be joined by a DNA ligase. Sticky-end fragments can be ligated not only to the fragment from which it was originally cleaved, but also to any other fragment with a compatible sticky end. The sticky end is also called a cohesive end or complementary end. If a restriction enzyme has a non-degenerate palindromic cleavage site, all ends that it produces are compatible. Ends produced by different enzymes may also be compatible. As used herein, compatible restriction sites also include those double-stranded sequences that, once cleaved by appropriate restriction enzymes, generate “blunt ends” that can be joined by a DNA ligase. As used in this application, compatible restriction sites are also referred to as generic restriction sites or universal restriction sites. As used herein, “restriction site” refers to specific sequences of nucleotides that are recognized by restriction endonucleases. Restriction endonucleases (restriction enzymes) generally recognize restriction sites that are palindromic and double stranded. In the context of the present application, restriction site sequences may be referred to as single stranded or double stranded.

In general, any restriction sites cleavable by any type 1, type 2 or type 3 restriction enzymes can be used for the invention. In some embodiments, any restriction sites cleavable by type 2 restriction enzymes can be used. The restriction enzymes and their recognition sequences are well known in the art. Exemplary restriction recognition sites are listed in Table 1. The sequences of suitable restriction sites can be incorporated into the polynucleotide linker sequence using standard recombinant technology.

The use of compatible restriction sites allows reformatting pools of ScFv to other molecular format independent of sequence information. This can be achieved using generic restriction sites which don't cut or don't cut frequently in the antibody VH or VL gene sequences and positioned at the 5′ or 3′ ends of VL and VH genes, or incorporated within a polynucleotide that encodes a flexible linker which is situated between the VH and VL genes. Pools of selected ScFv can thus be transferred for expression or display whilst maintaining VL and VH linkage during this transfer. The retention of VL and VH linkage during transfer is an important option to prevent the shuffling of V genes and loss of the selected binding pair during reformatting. Alternative cloning procedures that allow combinatorial shuffling (non linked transfer of VH and VL) may be used when extra diversity is desirable during transfer.

In some embodiments, compatible restriction sites suitable for the invention include restriction sites cleavable by restriction enzymes that don't cut or don't cut frequently in the antibody VH or VL gene sequences. For example, suitable compatible restriction sites can be any sites cleavable by restriction enzymes that cut, on average, less than 30%, 25%, 20%, 15%, 10%, 5%, 4%, 3%, 2%, 1%, 0.08%, 0.06%, 0.04%, 0.02%, 0.01%, or 0.005% of the population of VH or VL genes. The cutting frequency of restriction enzymes is dependent upon the nucleotide composition of the DNA source of the coding region. In some embodiment, the polynucleotide linker of the present invention include one or more compatible restriction sites cleavable by restriction enzymes that don't cut or don't cut frequently in antibody VH or VL genes including, but not limited to, ApaLI, AscI, AvaI, AvrII, MfeI, BamHl, BcII, BsiWI, BspEI, BssHII, BstEII, EcoRV, HindIII, Mlul, NcoI, NotI, XbaI, XhoI, Xmal, PciI, Pstl, NheI, SacI, Sall, SfiI and Smal. In some embodiments, the polynucleotide linker may contain restriction sites cleavable by any one of the above enzymes. For example, the polynucleotide linker may contain one or more compatible restriction sites cleavable by SfiI. In some embodiments, the polynucleotide linker may contain a first compatible restriction site cleavable by SfiI and a second compatible restriction site cleavable by SfiI, wherein the first and second compatible restriction sites are non compatible with each other. In some embodiments, the polynucleotide linker may contain a combination of restriction sites cleavable by any of the above enzymes, such as AscI and MfeI; AscI and SfiI; ApaLI and NotI; ApaLI and NheI; or ApaL 1 and BstEII. In other embodiments, the polynucleotide linker may contain a combination of restriction sites cleavable by any of the above enzymes, such as SfiI, BssHII, ApaLI or MfeI; XhoI, Sall, BcII, BstEII, Mlul, Smal or XbaI; XhoI, Sall, BspEi, ApaLI, BssHII or EcoRV; and SfiI, BclL, AvrII, BsiWI or BamHl. In yet other embodiments, the polynucleotide linker may contain a combination of restriction sites cleavable by any of the above enzymes, such as SfiI, BssHII or ApaLI; XhoI, Sall, BcII, SacI, AvrII, BsiWI or Mlul; MfeI, BspEI, ApaLI, BssHII, XhoI or Sall; and SfiI, BcII, XhoI, Sall or BstEII.

In some embodiments, a polynucleotide linker of the invention contain a combination of restriction sites that are present at fixed relative positions. For example, a polynucleotide linker may include a combination of compatible restriction sites including, from 5′ to 3′, an ApaLI site, a SacI site, an XhoI site and a SfiI Site. In some embodiments, a polynucleotide linker includes a combination of compatible restriction sites including, from 5′ to 3′, an ApaLI site, a SacI site, an XhoI site, BstEII, and a SfiI Site. In some embodiments, a polynucleotide linker includes a combination of compatible restriction sites including, from 5′ to 3′, an AscI site, a PciI site, a HindIII site, an ApaLI site, a SacI site, an AvaI site, an XhoI site, an MfeI site, an Xmal site, a Smal site, a BstEII site, a BcII site, and a SfiI site. One such exemplary polylinker is described in Example 3 and the nucleotide sequence of this exemplary polynucleotide is shown in SEQ ID NO:1. In some embodiments, a polynucleotide linker includes a combination of compatible restriction sites including, from 5′ to 3′, an SfiI, BssHII, ApaLI or MfeI site; an XhoI, Sall, BciI, BstEII, Mlul, Smal or XbaI site; an XhoI, Sall, BspEi, ApaLI, BssHII or EcoRV site; and an SfiI, BclL, AvrII, BsiWI or BamHl site. One exemplary polylinker includes, from 5′ to 3′, BssHI; XhoI; BspEI; and AvrII or BsiWI. In other embodiments, a polynucleotide linker includes a combination of compatible restriction sites including, from 5′ to 3′, an SfiI, BssHII or ApaLI site; an XhoI, Sall, BcII, SacI, AvrII, BsiWI or Mlul site; an MfeI, BspEI, ApaLI, BssHII, XhoI or Sall site; and an SfiI, BcII, XhoI, Sall or BstEII site. One exemplary polylinker includes, from 5′ to 3′, BssHI; AvrII or BsiWI; BspEI; and XhoI.

The nucleotide sequences between the compatible restriction sites can be changed. Thus, in some embodiments, a polynucleotide linker containing the same combination of restriction sites (i.e., from 5′ to 3′, an AscI site, a PciI site, a HindIII site, an ApaLI site, a SacI site, an AvaI site, an XhoI site, an MfeI site, an Xmal site, a Smal site, a BstEII site, a BcII site, and a SfiI site) can have a polynucleotide sequence at least 50%, 55%, 60%, 65%, 70%, 75%, 80%, 85%, 90%, 95%, 96%, 97%, 98%, 99%, or 99.5% identical to SEQ ID NO:1. In certain embodiments, the polynucleotide sequence includes a nucleotide sequence at least 95% identical to SEQ ID NO:1.

In some embodiments, a polynucleotide linker includes a combination of compatible restriction sites including, from 5′ to 3′, a PciI site, a HindIII site, an ApaLI site, a SacI site, an AscI site, an XhoI site, an AvaI site, an MfeI site, a BstEII site, and a SfiI site. One such exemplary polylinker is described in Example 5 and the nucleotide sequence of the exemplary polynucleotide is shown in SEQ ID NO:2.

The nucleotide sequences between the compatible restriction sites can be changed. Thus, in some embodiments, a polynucleotide linker containing the same combination of restriction sites (i.e., from 5′ to 3′, a PciI site, a HindIII site, an ApaLI site, a SacI site, an AscI site, an XhoI site, an AvaI site, an MfeI site, a BstEII site, and a SfiI site) can have a polynucleotide sequence at least 50%, 55%, 60%, 65%, 70%, 75%, 80%, 85%, 90%, 95%, 96%, 97%, 98%, 99%, or 99.5% identical to SEQ ID NO:2. In certain embodiments, the polynucleotide sequence includes a nucleotide sequence at least 95% identical to SEQ ID NO:2.

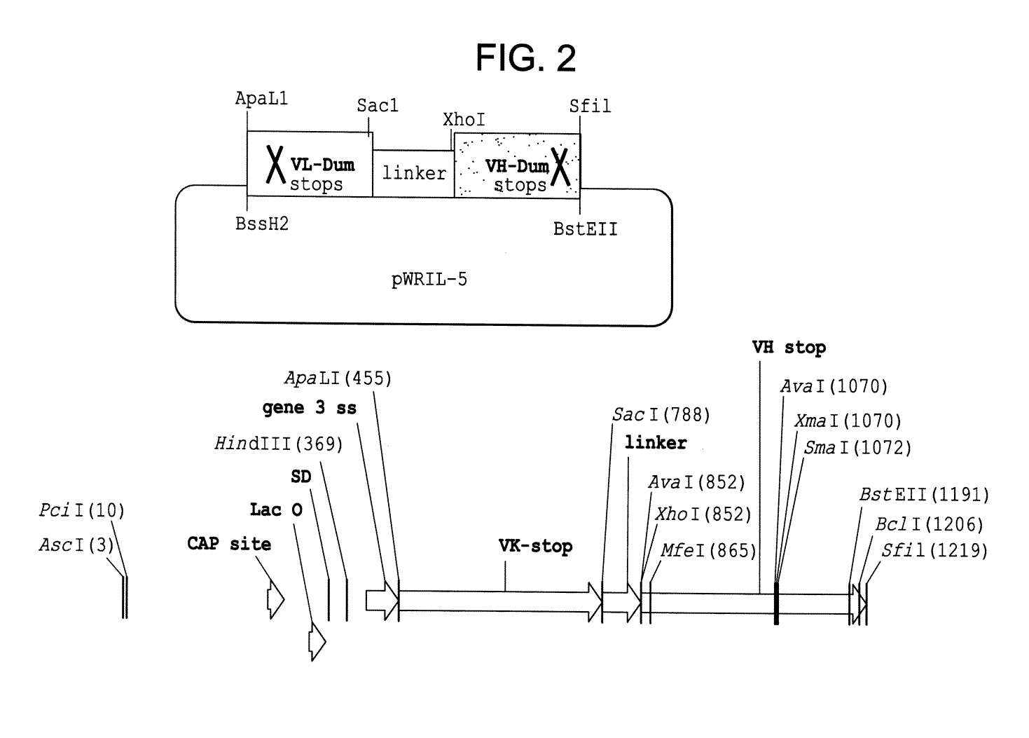

In some embodiments, a dummy VL gene or a dummy VH gene can be incorporated between the compatible restrictions sites. Dummy VL or VH genes can be constructed by introducing stop codons, typically in all three frames, into an existing VL or VH gene, respectively. Compatible restriction sites can be introduced at the 5′ and 3′ ends of the dummy VL and VH genes and cloned into a polynucleotide linker using compatible restriction sites. Dummy VH and VL genes can be replaced by VH and VL gene of interest, respectively, using compatible restriction sites as described below. An exemplary ScFv dummy construct is described in Example 3 and illustrated in FIG. 2.

Thus, the invention provide a modular polylinker design in which different elements (e.g., VH or VL gene) can be cloned and exchanged independently using compatible restriction sites present at fixed positions. The polylinker design also facilitates movement of large pools of VH and VL in a linked format using restriction-based cloning procedure into vectors with different format (e.g., Fabs and IgG) independent of sequence information. As described below, typically, reformatting involves two-step cloning procedure including transfer of both V-genes using outer restriction sites first, followed by replacement of the ScFv linker sequence with appropriate sequence fragments for either Fab or IgG expression.

TABLE 1
Exemplary restriction enzymes and corresponding
sites (single strand 5′ to 3′ shown)

| ENZYME | RECOGNITION SITE |
|---|---|
| Aat II | GACGI▾C |
| AccI | GT▾(A/T)(T/G)AC |
| AccIll | T▾CCGGA |
| Acc65 I | G▾GTACC |
| AccB7 I | CCANNNN▾NTGG |
| | (SEQ ID NO: 4) |
| AcyI | G(A/G)▾CG(T/C)C |
| Age l | A▾CCGGT |
| Alu l | AG▾CT |
| A/w26 I | G▾TCTC(1/5) |
| A/w44 I | G▾TGCAC |
| Apa l | GGGCC▾C |
| Ava I | C▾(T/C)CG(A/G)G |
| Ava lI | G▾G(A/T)CC |
| Ba/ I | TGG▾CCA |
| BamH l | G▾GATCC |
| Ban l | G▾G(T/C)(A/G)CC |
| Ban II | G(A/G)GC(T/C)▾C |
| Bbu l | GCATG▾C |
| Bc/ I | T▾GATCA |
| Bgl l | GCCNNNN▾NGGC |
| | (SEQ IOD NO: 5) |
| Bg/ Il | A▾GATCT |
| BsaM I | GATTGCN▾ |
| BsaO I | CG(A/G)(T/C)▾CG |
| Bsp1286 I | G(G/A/T)GC(C/A/T)▾C |
| BsrBR I | GATNN▾NNATC |
| | (SEQ ID NO: 6) |
| BsrS I | ACTGGN▾ |
| BssH II | G▾CGCGC |
| Bst71 I | GCAGC(8/12) |
| Bst98 I | C▾TTAAG |
| Bst E II | G▾GTNACC |
| Bst O I | CC▾(A/T)GG |
| Bst X I | CCANNNNN▾NTGG |
| | (SEQ ID NO: 7) |
| Bst Z I | C▾GGCCG |
| Bsu36 I | CC▾TNAGG |
| Cfo I | GCG▾C |
| Cla l | AT▾CGAT |
| Csp I | CG▾G(A/T)CCG |
| Csp 45 I | TT▾CGAA |
| Dde I | C▾TNAG |
| Dpn I | G^{me}A▾TC |
| Dra I | TTT▾AAA |
| EclHK I | GACNNN▾NNGTC |
| | (SEQ ID NO: 8) |
| Eco47 III | ACG▾GCT |
| Eco52 I | C▾GGCCG |
| Eco72 I | CAC▾GTG |
| EcoI CR I | GAG▾CJC |
| EcoR I | G▾AATTC |
| EcoR V | GAT▾ATC |
| Fok I | GGATG(9/13) |
| Hae ll | (A/G)GCGC▾(T/C) |
| HaelIl | GG▾CC |
| Hha I | GCG▾C |
| Hinc II | GT(T/C)▾(A/G)AC |
| Hind III | A▾AGCTT |
| Hinf I | G▾ANTC |
| Hpa I | GTT▾AAC |
| Hpa II | C▾CGG |
| Hsp92 I | G(A/G)▾CG(T/C)C |
| Hsp92 II | CATG▾ |
| I-Ppo I | CTCTCTTAA▾GGTAGC |
| | (SEQ ID NO: 9) |
| Kpn I | GGTAC▾C |
| Mbo I | ▾GATC |
| Mbo II | GAAGA(8/7) |
| Mlu l | A▾CGCGT |
| Msp I | C▾CGG |
| MspA I | C(A/C)G▾C(G/T)G |
| Nae l | GCC▾GGC |
| Nar | GG▾CGCC |
| Nci I | CC▾(G/C)GG |
| Nco I | C▾CATGG |
| Nde l | CA▾TATG |
| NgoM I | G▾CCGGC |
| Nhe I | G▾CTAGC |
| Not I | GC▾GGCCGC |
| Nru I | TCG▾CGA |
| Nsi l | ATGCA▾T |
| Pst l | CTGCA▾G |
| Pvu l | CGAT▾CG |
| Rvu II | CAG▾CTG |
| Rsa l | GT▾AC |
| Sac I | GAGGCT▾C |
| Sac II | CCGC▾GG |
| Sal l | G▾TCGAC |
| Sau3A I | ▾GATC |
| Sau96 I | G▾GNCC |
| Sca l | AGT▾ACT |
| Sfi I | GGCCNNNN▾NGGCC |
| | (SEQ ID NO: 10) |
| Sgf I | GCGAT▾CGC |
| Sin I | G▾G(A/T)CC |
| Sma l | CCC▾GGG |
| SnaB I | TAC▾GTA |
| Spe l | A▾CTAGT |
| Sph I | GCATG▾C |
| Ssp l | AAT▾ATT |
| Stu l | AGG▾CCT |
| Sty l | C▾C(A/T)(T/A)GG |
| Taq I | T▾CGA |
| Tru9 I | T▾TAA |
| Tthlll I | GACN▾NNGTC |
| Vsp I | A▾TAAT |
| Xba I | T▾CTAGA |
| Xho I | C▾TCGAG |
| Xho II | (A/G)▾GATC(T/C) |
| Xma l | C▾CCGGG |
| Xmn I | GAANN▾NNTTC |
| | (SEQ ID NO: 11) |

### Phage Display Vectors

Typically, a phage display vector of the present invention is a vector containing phage derived nucleic acid sequences capable of expressing or conditionally expressing a heterologous polypeptide, for example, as a fusion protein with a phage protein (e.g., a phage surface protein). In some embodiments, a phage display vector of the present invention is a vector derived from a filamentous phage (e.g., phage f1, fd, and M13) or a bacteriophage (e.g. T7 bacteriophage and lambdoid phages. The filamentous phage and bacteriophage are described in e.g., Santini (1998) J. Mol. Biol. 282:125-135; Rosenberg et al. (1996) Innovations 6:1-6; Houshmand et al. (1999) Anal Biochem 268:363-370).

In particular, a phage display vector of the invention can include the following elements: (1) a promoter suited for constitutive or inducible expression (e.g., lac promoter); (2) a ribosome binding site and signal sequence preceding the sequence encoding displayed peptide; and (3) a polynucleotide linker containing compatible restriction sites as described above; (4) optionally, a tag sequence such as a stretch of 5-6 histidines or an epitope recognized by an antibody; (5) a suppressible codon (e.g., a termination codon); and (6) a sequence encoding a phage surface protein positioned in-frame to form a fusion to the peptide to be displayed.

In general, a phage display vector of the invention contains a promoter and/or regulatory region operably linked to a nucleic acid sequence encoding the heterologous polypeptide and a sequence encoding a phage surface protein. The term “operably linked” refers to a functional linkage between nucleic acid sequences such that the linked promoter and/or regulatory region functionally controls expression of the coding sequence. It also refers to the linkage between coding sequences such that they may be controlled by the same lined promoter and/or regulatory region. Such linkage between coding sequences may also be referred to as being linked in frame or in the same coding frame such that a fusion protein comprising the amino acids encoded by the coding sequences may be expressed.

In other embodiments of the invention, the ability to express a fusion protein is regulated in part by use of a regulated promoter or other regulatory region (e.g., an inducible promoter such that in the absence of induction, expression controlled by them is low or undetectable). Non-limiting examples of inducible promoters include the lac promoter, the lac UV5 promoter, the arabinose promoter, and the tet promoter. In some embodiments, an inducible promoter can be further restricted by incorporating repressors (e.g., lacI) or terminators (e.g., a tHP terminator). For example, repressor lacI and be used together with the Lac promoter. In some embodiments, a strong tHP terminator can be additionally inserted between the lacI gene and the Lac promoter.

As used herein, the term “phage surface protein” refers to any protein normally found at the surface of a filamentous phage (e.g., phage f1, fd, and M13) or a bacteriophage (e.g., λ, T4 and T7) that can be adapted to be expressed as a fusion protein with a heterologous polypeptide and still be assembled into a phage particle such that the polypeptide is displayed on the surface of the phage. Suitable surface proteins derived from filamentous phages include, but are not limited to, minor coat proteins from filamentous phages, such as gene. III proteins, and gene VIII proteins, major coat proteins from filamentous phages, such as, gene VI proteins, gene VII proteins, gene IX proteins, gene 10 proteins from T7, and capsid D protein (gpD) of bacteriophage λ. In some embodiments, a suitable phage surface protein is a domain, a truncated version, a fragment, or a functional variant of a naturally-occurring surface protein. For example, a suitable phage surface protein can be a domain of the gene III protein, e.g., the anchor domain or “stump.” Additional exemplary phage surface proteins are described WO 00/71694, the disclosures of which are hereby incorporated by reference. As appreciated by the skilled artisan, the choice of a phage surface protein is to be made in combination with a consideration of the phage display vector and the cell to be used for propagation thereof.

The displayed polypeptide is typically covalently linked to the phage surface protein. The linkage results from translation of a nucleic acid encoding the polypeptide component fused to the surface protein. The linkage can include a flexible peptide linker, a protease site, or an amino acid incorporated as a result of suppression of a stop codon.

Suppressible codons are known in the art. For example, suppressible codons can be termination codons including UAG (referred to as the amber codon), UAA (referred to as the ochre codon), and UGA. UAG, UAA and UGA indicate the mRNA codon. The corresponding nucleotide sequences present in the vector are TAG, TAA and TGA, respectively. The choice of termination codon can also be augmented by introduction of particular sequences around the codon.

A specific initiation signal may be incorporated to further regulate translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. A low efficiency ribosome-binding sequence or translation initiation signal may be used to further decrease protein production without induction.

Any peptide sequences capable of driving or directing secretion of expressed protein or polypeptide can be used as leader sequences for the phage display vectors. Exemplary leader sequences include, but not limited to, a PelB leader sequence and an Omp A leader sequence.

In addition, optionally, a fusion polypeptide can include a tag that may be useful in purification, detection and/or screening. Suitable tags include, but not limited to, FLAG, poly-his, gD tag, c-myc, fluorescence protein or β-galactosidase.

General methods for constructing phage display vectors, phage display libraries and the method of use are described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol. Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Exemplary phage display vectors of the invention are described in the Examples section below.

### Ribosome Display Vectors

The ribosome display vectors of the present invention include vectors suitable for prokaryotic or eukaryotic display system. A prokaryotic ribosome display system is also referred to as polysome display system.

A ribosome display vector of the invention typically includes a promoter or RNA polymerase binding sequence, a ribosome binding site, a translation initiation sequence, an amino acid spacer sequence separating the displayed peptide from ribosome to assist correct folding of the peptide and a polynucleotide linker containing one or more compatible restriction sites as described above. Optionally, the ribosome display vector may also include one or more sequences encoding detection tags, 3′ stem loop structure and/or 5′ stem loop structure to protect synthesized mRNA, a translation enhancer or “activator” sequence(s). Typically, the ribosome display vector of the invention lacks a stop codon in-frame of the displayed polypeptide.

The promoter or RNA polymerase binding sequence suitable for the invention may include any promoters suitable for in vitro translation. Exemplary promoters include, but are not limited to, T7, T3, or SP6 promoters, or any sequences recognized by RNA polymerases T7, T3 or SP6.

In some embodiments, a ribosome display vector of the invention may include two promoters, such as both the T7 and SP6 promoters. A ribosomal binding site may be positioned downstream of or within the promoter region. This ribosome binding site may be specific for prokaryotic ribosomal complexes (such as a Shine-Dalgarno sequence) if a prokaryotic translation procedure is used. Suitable prokaryotic translation systems include, but are not limited to E. coli S30 system. The ribosome binding site may also be specific for a eukaryotic translation system (such as Kozak consensus sequence), if a eukaryotic translation procedure is used. Suitable eukaryotic translation systems include, but are not limited to, the rabbit reticulocyte system (Krawetz et al., Can. J. Biochem. Cell. Biol. 61:274-286, 1983; Merrick, Meth. Enzymol. 101:38, 1983). One exemplary Kozak consensus sequence is GCCGCCACCATGG (SEQ ID NO:12).

Additional translation enhancer or “activator” sequences may also be included. For example, the translation enhancer of X. leavis β globin gene may be inserted between the promoter and translation initiation site. Other exemplary translation enhancers or activator sequences include, but are limited to, untranslated “leader sequences” from tobacco mosaic virus (Jobling et al. Nucleic Acids Res. 16:4483-4498, 1988), 5′ untranslated region from alfalfa mosaic virus RNA 4 (Jobling and Gehrke, Nature 325:622-625, 1987), black beetle virus (Nodavirus) RNA 2 (Friesen and Rueckert, J. Virol. 37:876-886, 1981), and turnip mosaic virus, and brome mosaic virus coat protein mRNAs (Zagorski et al., Biochimie 65:127-133, 1983).

An amino acid spacer sequence can be engineered at the C-terminus to separate the displayed peptide from ribosome. Without wishing to be bound by any theories, it is contemplated that the spacer sequence allows the displayed polypeptide to exit completely from the ribosome “tunnel” and to fold correctly. Typically, a suitable spacer sequence encodes at least 20 amino acids in length. In particular, a suitable spacer length may include at least 30 amino acids, 40 amino acids, 50 amino acids, 60 amino acids, 70 amino acids, 80 amino acids, 90 amino acids, 100 amino acids. In certain embodiments, the spacer includes 23 amino acids. In certain embodiments, the spacer includes 69 amino acids. In certain embodiments, the spacer includes 116 amino acids. Suitable spacer sequences can be derived from any known proteins, such as, for example, the constant region of immunoglobulin kappa chain (C1c), gene III of filamentous phage M13, and the CH3 domain of human IgM.

A tag sequence may be incorporated into the ribosome vector of the invention. Typically, the tag sequence is incorporated at the N terminus or C terminus of the displayed peptide. Suitable tags include, but are not limited to, a stretch of histidines (e.g., 5-6 histidines), an epitope recognized by an antibody (e.g., substance P or Flag).

The ribosome display vector may also include a 3′ region with palindromic sequences that may form a stem loop structure. Without wishing to be bound by any theories, the stem loop structure may impede translocation, thus, palindromic sequences slow down the movement of ribosomes during translation and prevent ribosomes from “falling off” the mRNA and thereby protecting synthesized mRNA and increasing the number of polysomes in the in vitro translation step. Similarly, the ribosome display vector may also include a 5′ stem loop structure.

In addition, the 3′ region may contain a poly-A or other polynucleotide stretch for later purification of the mRNA from the in vitro translation reaction by hybridization to a complementary homopolymeric sequence.

The ribosome display vector may be chemically synthesized by protocols well known to those skilled in the art. Alternatively, each of the above elements may be incorporated into one or more plasmids, amplified in microorganisms, purified by standard procedures, and cut into appropriate fragments with restriction enzymes before assembly into the vector. General methods for constructing ribosome display vectors, ribosome display libraries and method of use are described in U.S. Pat. Nos. 5,643,768, 5,658,754, and 7,074,557, and in Mattheakis et al., (1994) PNAS USA 91, 9022 9026; Mattheakis et al., (1996) Methods Enzymol. 267, 195 207; Gersuk et al., (1997) Biotech and Biophys. Res. Corn. 232, 578 582; Hanes and Pluckthun (1997) PNAS USA 94, 4937 4942; Hanes et al., (1998) PNAS USA 95, 14130 50; He and Taussig (1997) NAR 5132 5234, the teachings of all of which are hereby incorporated by reference.

### Collection

As used herein, the phrase “collection of genes,” as in e.g. collection of VH genes or collection of VL genes, “collection of nucleic acids,” “collection of polynucleotides,” and “collection of polypeptides” is a population of diverse variants, for example, nucleic acid variants which differ in nucleotide sequence or polypeptide variants which differ in amino acid sequence.

### Library

As used herein, the term “library” refers to a mixture of heterogeneous polypeptides or nucleic acids. Typically, a library includes a plurality of members, each of which contains a polypeptide or nucleic acid sequence. Typically, each polypeptide or nucleic acid sequence is incorporated into a vector. A library according to the invention typically encompasses a collection of polypeptides or nucleic acids. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. As used herein, the term “organism” refers to all cellular life-forms, such as prokaryotes and eukaryotes, as well as non-cellular, nucleic acid-containing entities, such as bacteriophage and viruses.

In particular, antibody libraries can incorporate diversity from a variety of sources, including from synthetic nucleic acid, naive nucleic acids, patients (e.g., immunized or diseased human subjects), and animals (e.g., immunized animals).

In some embodiments, immune cells can be used as a natural source of diversity for the variation of antibodies, MHC-complexes and T cell receptors. Some examples of immune cells are B cells and T cells. The immune cells can be obtained from, e.g., a human, a primate, mouse, rabbit, camel, or rodent. The cells can be selected for a particular property. For example, T cells that are CD4+ and CD8− can be selected. B cells at various stages of maturity can be selected.

Naturally diverse sequences can be obtained as cDNA produced from total RNAs isolated from cell and samples obtained from a subject, e.g., Peripheral Blood Leucocytes (PBL's). The reverse transcription of the first (antisense) strand can be done in any manner with any suitable primer. See, e.g., de Haard et al. (1999) J. Biol. Chem. 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. Poly-dT primers (e.g., for the heavy-chain genes) or synthetic primers that hybridize to a synthetic sequence ligated to the mRNA strand may also be used. cDNA can be amplified, modified, fragmented, or cloned into a vector to form an antibody library. See, e.g., de Haard et al. (1999) supra.

### Constructing ScFv Library

Typically, construction of an ScFv library involves two steps. The first step involves isolating and cloning the VH and VL genes separately from an RNA sample isolated from a subject of interest as described above. Desirable compatible restriction sites can be incorporated by proper primer design using methods well known in the art. More than one round of PCR reactions can be used to amplify the VH and VL genes or to introduce desirable restriction sites. VH and VL gene can be cloned into respective dummy vectors using appropriate restriction sites to construct VH or VL gene only collection. The second step involves isolating the collection of VL genes by digestion with compatible restriction enzymes and transferring the VL collection into the VH gene only collection to construct an ScFv library.

In some embodiments, a method for constructing a single chain Fv (ScFv) library of the invention includes the steps of: (1) introducing an XhoI restriction site to the 5′ end and a SfiI or BstEII restriction site to the 3′ end of a collection of VH genes; (2) cloning the collection of VH genes into a plurality of vectors using a first restriction site compatible with XhoI and a second restriction site compatible with Sfi1 or BstEII; (3) introducing a SacI restriction site to the 5′ end and an ApaLI restriction site to the 3′ end of a collection of VL genes; and (4) cloning the collection of VL genes into the plurality of vectors using restriction sites compatible with ApaL1 and Sac1.

In some embodiments, step (1) includes synthesizing first strand cDNA from isolated total RNA and amplifying VH genes by PCR amplification using one or more primer sets including a forward primer containing an XhoI restriction site and a reverse primer containing a SfiI or BstEII restriction site.

In some embodiments, step (3) includes synthesizing first strand cDNA from isolated total RNA and amplifying VL genes by PCR amplification using one or more primer sets including a forward primer containing an ApaL1 restriction site and a reverse primer containing a Sac1 restriction site.

Exemplary primers and methods for constructing ScFv libraries are described in the Examples section.

### Target

The target is a ligand for which a specific binding member or members of the collection is to be identified. Where the members of the collection are antibody molecules, the target may be an antigen or an epitope and where the members of the collection are enzymes, the target may be a substrate.

### Selections

The selection process can be performed manually or using an automated method. In some cases, non-specific binding and other non-ideal properties require more than one selection cycle. Additional selection cycles increase the enrichment for candidate library members. To repeat a selection step, eluted library members are amplified then reapplied to the target. Depending on the implementation, different numbers of selection cycles may be sufficient to identify a pool of candidate library members from a library having a vast diversity. For example, one, or two rounds of selection may be sufficient.

Some exemplary selection processes are as follows.

Panning. The target molecule is immobilized to a solid support such as a surface of a microtitre well, matrix, particle, or bead. The display library is contacted to the support. Library members that have affinity for the target are allowed to bind. Non-specifically or weakly bound members are washed from the support. Then the bound library members are recovered (e.g., by elution) from the support. Recovered library members are collected for further analysis (e.g., screening) or pooled for an additional round of selection.

Magnetic Particle Processor. One example of an automated selection uses magnetic particles and a magnetic particle processor. In this case, the target is immobilized on the magnetic particles. The KingFisher™ system, a magnetic particle processor from Thermo LabSystems (Helsinki, Finland), is used to select display library members against the target. The display library is contacted to the magnetic particles in a tube. The beads and library are mixed. Then a magnetic pin, covered by a disposable sheath, retrieves the magnetic particles and transfers them to another tube that includes a wash solution. The particles are mixed with the wash solution. In this manner, the magnetic particle processor can be used to serially transfer the magnetic particles to multiple tubes to wash non-specifically or weakly bound library members from the particles. After washing, the particles are transferred to a tube that includes an elution buffer to remove specifically and/or strongly bound library members from the particles. These eluted library members are then individually isolated for analysis or pooled for an additional round of selection. Detailed magnetic particle processor selection processes are described in U.S. Application Publication No. 20030224408.

Cell-Based Selections. The selection can be performed by binding the display library to target cells, and then selecting for library members that are bound by the cells. Cell-based selections enable the identification of ligands that recognize target molecules as presented in their natural milieu, e.g., including post-translational modifications, associated proteins and factors, and competing factors. Further, since cell-based selections are not directed against a specific singular target molecule, no a priori information is required about the target. Rather, the cell itself is a determinant. Later steps, particular functional assays, can be used to verify that identified ligands are active in targeting effector functions to the cell. Detailed cell-based selection processes are described in U.S. Application Publication No. 20030224408.

In vivo Selections. The selection can be done in vivo to identify library members that bind to a target tissue or organ, e.g., as described in Kolonin et al. (2001) Current Opinion in Chemical Biology 5:308-313, Pasqualini and Ruoslahti (1996) Nature 380:364-366, and Paqualini et al. (2000) “In vivo Selection of Phage-Display Libraries” In Phage Display: A Laboratory Manual Ed. Barbas et al. Cold Spring Harbor Press 22.1-22.24. For example, a phage display library is injected into a subject (e.g., a human or other mammal). After an appropriate interval, a target tissue or organ is removed from the subject and the display library members that bind to the target site are recovered and characterized.

### Affinity Maturation/Optimization

In some embodiments, after initial selection using a first library, a selected population of library members can be mutagenized to improve the binding affinity or any other properties of the selected members. For example, a first display library is used to identify one or more ligands for a target (also known as lead identification). These identified ligands are then mutated to form a second display library. Additional diversity are introduced by mutagenesis. Higher affinity ligands are then selected from the second library, e.g., by using higher stringency or more competitive binding and washing conditions. This process is known as affinity maturation or optimization.

In some embodiments, a phage display library of the present invention is used for initial identification of target-binding polypeptides. The selected pool of nucleic acid fragments encoding the target-binding polypeptides can be retrieved by digestion using restriction enzymes that cleave one or more compatible restriction sites. The retrieved fragments can then be cloned “en masse” into ribosome display vectors of the present invention using one or more compatible restriction sites. The ribosome display vectors containing the selected nucleic acid fragments transferred from the phage display library can be further mutagenized to form a second library, e.g., a ribosome display library. The diversity of a ribosome display library can be up to more than 10¹².

Numerous techniques can be used to mutate the identified initial ligands to introduce further diversity. These techniques include, but are not limited to, error-prone PCR (Leung et al. (1989) Technique 1:11-15), recombination, DNA shuffling using random cleavage (Stemmer (1994) Nature 389-391), RACHITT™ (Coco et al. (2001) Nature Biotech. 19:354), site-directed mutagenesis (Zoller et al. (1987) Methods Enzymol. 1987; 154:329-50.; Zoller et al. (1982) Nucl. Acids Res. 10:6487-6504), cassette mutagenesis (Reidhaar-Olson (1991) Methods Enzymol. 208:564-586) and incorporation of degenerate oligonucleotides (Griffiths et al. (1994) EMBO J. 13:3245).

For antibodies, mutagenesis can be directed to the CDR regions of the heavy or light chains. In some embodiments, mutagenesis can be directed to framework regions near or adjacent to the CDRs.

Methods for identification of the members of the ribosome display library with desirable binding affinity or other properties and retrieving the nucleic acid sequences encoding the selected polypeptides are well known in the art. For example, exemplary methods are described in U.S. Pat. Nos. 5,643,768, 5,658,754, and 7,074,557.

### Reformatting

Following selection and identification of a library member containing a nucleic acid sequence encoding a displayed polypeptide with desirable properties, the nucleic acid can be retrieved from the display vector and transferred to an expression vector for production or further analysis. This process is typically known as reformatting. Thus, the reformatting process is used, for example, to transfer nucleic acid from a display vector to a vector suitable for bacteria or mammalian cell production. In one embodiment, each selected library member is reformatted individually. In another embodiment, the library members are combined and reformatted en masse.

The reformatting process can be tailored to the expression system used initially for display and for the secondary expression system. For example, the reformatting process is particularly important for the analysis of ribosome display products because typical ribosome vectors are not compatible with bacterial or mammalian expression system, while the same phage display vector can be used to express the selected displayed polypeptide in a bacteria expression system.

In some embodiments, the selected ScFv polypeptide can be reformatted into other immunoglobulin formats including, but not limited to, IgG, ScFv-Fc fusions, F(ab′)₂, F(ab)₂, Fab′, Fab, diabodies, triabodies or tetrabodies. In one example of en masse reformatting, the reformatting of ScFv involves a two-step process. The first cycle includes digesting display vectors to release nucleic acid fragments that include minimally a light chain variable coding region and a heavy chain variable coding region using for example, compatible restriction sites. The fragments are cloned into a vector for mammalian expression. During this step, the transfer of the nucleic acid fragments encoding both VH and VL genes insures that combinations of heavy and light chain present in the display vector are maintained in the expression vector. Further, the transfer process can be used to switch from a prokaryotic promoter to a mammalian promoter on the 5′ end of the coding strand and from a sequence encoding a bacteriophage coat protein (or fragment thereof) to a sequence encoding an Fc domain on the 3′ end of the coding strand. General methods for cloning are described in standard laboratory manuals, e.g., Sambrook et al. (2001) Molecular Cloning: A Laboratory Manual (Third Edition), Cold Spring Harbor Laboratory Press.

In the second step, the region intervening between the light chain coding region and the heavy chain-coding region is substituted. For example, the linker region between VH and VL genes can be replaced with a sequence that includes a prokaryotic ribosome binding site (RBS), or a sequence with an internal ribosomal entry site (IRES) or a sequence including a eukaryotic promoter. Also in this process, signals for secretion (e.g., the prokaryotic or eukaryotic signals for secretion) and sequences from the constant regions of the immunoglobulin molecules (e.g., Ck, CH1) can be inserted. In some implementations, the intervening region is substituted by recombination in a cell. In still others, the intervening region is not substituted, but rather sequences are inserted (e.g. using site-specific recombination) without excising e.g. the sequences designed for prokaryotic expression.

Hybrid signal sequences that are functional in both prokaryotic and eukaryotic cells can be used to obviate reformatting of some (e.g., at least the 3′ region of the signal sequence, e.g., the −3, −2, and −1 positions) or all of the signal sequence. In some cases, a signal sequence is functional in multiple expression systems (e.g., both pro- and eukaryotic systems). For example, the signal sequence of some bacterial beta-lactamases is functional in eukaryotic cells and prokaryotic cells. See, e.g., Kronenberg et al., 1983, J. Cell Biol. 96, 1117-9; Al-Qahtani et al., 1998, Biochem. J. 331, 521-529. Signal sequences that function in multiple hosts can also be designed on the basis of the requirement of such signal sequence (consensus rules) in the respective expression hosts, or may be selected empirically.

In some embodiments, the selected ScFv polypeptide of the invention can be reformatted to small modular immunopharmaceutical (SMIP™) drug format (Trubion Pharmaceuticals, Seattle, Wash.) using similar cloning strategy. SMIPs are single-chain polypeptides composed of a binding domain for a cognate structure such as an antigen, a counter receptor or the like, a hinge-region polypeptide having either one or no cysteine residues, and immunoglobulin CH2 and CH3 domains (see also www.trubion.com). The SMIP drug designs are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Encoding nucleic acid, whether reformatted or not, may be used in production of the encoded polypeptide or peptide using any technique available in the art for recombinant expression.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antibody fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of a specific binding member, see for recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

Thus, nucleic acid encoding a specific polypeptide selected using a method of the invention, or a component of such a specific polypeptide (e.g. VH and/or VL domain) may be provided in an expression system for production. This may comprise introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

The introduction may be followed by causing or allowing expression from the nucleic acid, e.g. by culturing host cells under conditions for production of the encoded product. The present invention also provides a method which comprises using a construct as stated above in an expression system in order to express a specific binding member or polypeptide as above.

Following production by expression, a product may be isolated and/or purified and may be formulated into a composition comprising at least one additional component. Such a composition may comprise a pharmaceutically acceptable excipient, vehicle or carrier.

Further aspects and embodiments of the present invention will be apparent to those skilled in the art in the light of the present disclosure. It should further be noted that all documents mentioned anywhere herein are incorporated by reference.

The present invention will now be further illustrated with reference to the following experimental examples.

### Example 1

### ScFv Library Design

A ScFv library was designed using generic restriction sites which facilitate “bulk” reformatting of ScFv antibodies into other immunoglobulin formats, such as, for example, IgG, Fab, ScFv-Fc fusions, small modular immunopharmaceutical (SMIP™), and other single chain antibodies, such as e.g., nanobodies and shark antibodies (see respectively US Pat. App. Pub. No. 20080107601 and International Pat. App. Pub. No. WO 03/014161, which are incorporated herein by reference.)

A ScFv library was designed using generic restriction sites which facilitate “bulk” reformatting of ScFv antibodies into other immunoglobulin formats, such as, for example, IgG, Fab, ScFv-Fc fusions, small modular immunopharmaceutical (SMIP™.)

In this experiment, the vector designs and reformatting procedures took into consideration the following factors:

(1) To allow retention of selected VL and VH pairing;

(2) To allow reformatting without altering V gene sequence;

(3) To use restriction sites infrequent in germline V-genes;

(4) To allow reformatting independent of sequence information;

(5) To have modular design so that different elements can be independently exchanged.

Vectors suitable for ScFv format and compatible with Fab, IgG, SMIP™, and other mammalian expression/display vectors were designed. Important features of the ScFv vectors are discussed below.

First, vectors were designed to express an ScFv antibody in a VL-VH format because the VL-VH format is the preferred format for ScFv-Fc fusions or SMIPs. Such ScFv design allows direct reformatting to SMIPs (i.e., ScFv can be transferred to compatible SMIP vector directly) for high throughput protein expression.

Second, restriction sites that do not cut or cut rarely in germline V genes were introduced to the 5′ and 3′ ends of VH and VL genes. For example, an XhoI restriction site was introduced to the 5′ end of VH gene because Xho1 does not cut any human germline VH genes and cuts in 3 out of 40 (i.e., 7.5%) germline Vk genes. The Xho1 site can be incorporated at the 3′ end of the flexible linker (DGGGSGGGGSGGGGSS[SEQ ID NO:3]). The linker design is described below. A Sac1 site can be incorporated into 3′ end of VL gene (i.e., Jk segment). Sac1 site is present in only 1 out of 40 (i.e., 2.5%) germline Vk genes.

A flexible linker between the VL and VH genes was designed as illustrated below:

| | |
|---|---|
| Xhol | |
| ~~~~~· | |
| Asp Gly Gly Gly Ser Gly Gly Gly Gly Ser Gly Gly Gly Gly Ser Ser | (SEQ ID NO: 3) |
| GAT GGC GGT GGA TCG GGC GGT GGT GGA TCT GGA GGA GGT GGC TCG AGC | (SEQ ID NO: 13) |

As shown, this linker encodes 16 amino acids (DGGGSGGGGSGGGGSS [SEQ ID NO:3]) including two serines at the 3′ end, which incorporates an XhoI site. This linker is compatible for SMIP.

### Example 2

### V Gene Amplification Strategy

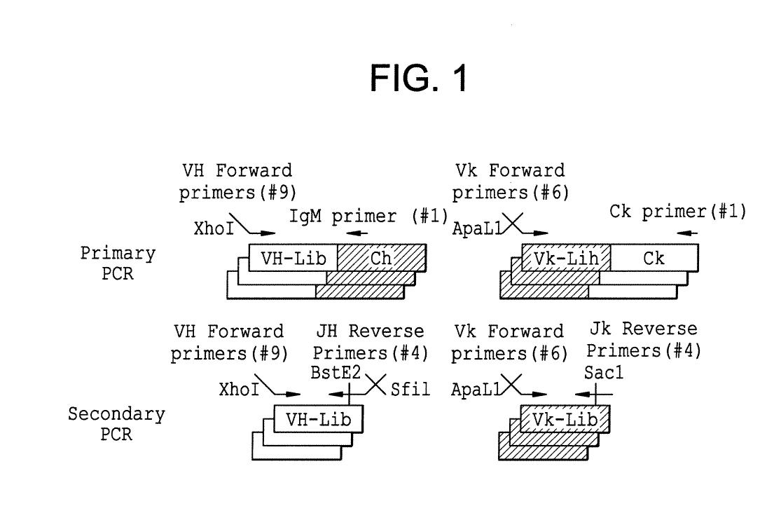

V gene amplification strategy is illustrated in FIG. 1. In order to introduce universal restriction sites to the 5′ and 3′ end of the VH and VL gene, PCR amplifications using primer sets incorporating desirable restriction sites are used. Typically, more than one round of PCR reactions are conducted. For example, VH collection can be amplified by a primary PCR and a secondary PCR. The primary PCR uses a VH forward primer containing an XhoI site and an IgM specific reverse primer. The secondary PCR uses the VH forward primer containing the XhoI site and a JH reverse primer containing an Sfi1 site and/or BstEII site.

As discussed above, Xho1 does not cut any human germline VH genes and only cuts in 3 out of 40 germline Vk genes. An exemplary design of a linker sequence incorporating an XhoI site at the 3′ end shown above.

BstEII cuts in 4 out of 51 (i.e., 7.8%) germline VH genes and 0 out of 40 germline Vk genes. An exemplary design of BstEII site in the FW4 region of VH gene is illustrated below:

| | |
|---|---|
| T L V T V S S | (SEQ ID NO: 14) |
| ACC CTG GTC ACC GTC TCC TAC end of Vh | (SEQ ID NO: 15) |
| BstEII | |

At least 5 different primers were designed to cover JH1-5 regions.

Similarly, VL collection can be amplified by a primary PCR and a secondary PCR. The primary PCR uses a Vk forward primer containing an ApaL1 site and a Ck specific reverse primer and the secondary PCR uses the Vk forward primer containing the ApaL1 site and a Jk reverse primer incorporating a Sac1 site. A linker sequence can be incorporated into the reverse primer for the secondary PCR.

ApaL1 does not cut in germline Vk genes and only cuts in 2 out of 51 (i.e., 3.9%) germline VH genes. An exemplary design of 5′ of VL gene is shown below to illustrate how a 5′ ApaLI site can be introduced to 5′ of VL gene.

| | |
|---|---|
| Ser His Ser Ala Gln | (SEQ ID NO: 16) |
| . . . end of Sig seq . . . TCT CAC AGT GCA CAG . . . Start of VL | (SEQ ID NO: 17) |
| ApaL1 | |

At least 6 different primers were designed to cover all Vk gene families (see Table 2).

Sad cuts in 1 out of 40 (i.e., 2.5%) germline Vk and 11 out of 51 (i.e., 21.6%) germline VH genes. As shown below, a SacI site can be introduced by converting a germline Jk sequence TKVEIKR (SEQ ID NO:18) to TKVELKR (SEQ ID NO:19) in the FW4 region.

| | | |
|---|---|---|
| Convert: | T K V E I K R (SEQ ID NO: 18: Germline Jk sequence) | |
| To: | T K V E L K R | (SEQ ID NO: 19: Conservative change of I to L) |
| | T K V E L K R | (SEQ ID NO: 19) |
| | ACC AAG CTG GAG CTC AAA CGT (SEQ ID NO:20) | |
| | Sac1 | |

At least 5 different primers were designed to cover Jk1-5 regions.

Additional exemplary primers suitable for the amplification of VH and VL genes are illustrated in Table 2.

TABLE 2
PRIMARY PCR PRIMERS

HuIgM-Rev (SEQ ID NO: 21)
5′ TGG AAG AGG CAC GTT CTT TTC TTT
HuCk-Rev (SEQ ID NO: 22)
5′ ACA CTC TCC CCT GTT GAA GGT CTT

VH Forward primers (Xho1 site is underlined)

HuVH1b/7A-For (SEQ ID NO: 23)
5′ ggc tat ggt tgc GGC TCG AGC CAG RTG CAG GTG GTG CAR TGT GG
HuVH1C-For (SEQ ID NO: 24)
5′ ggc tat ggt tgc GGC TCG AGC SAG GTC CAG CTG GTR CAG TCT GG
HuVH2-For (SEQ ID NO: 25)
5′ ggc tat ggt tgc GGC TCG AGC GAG RTC ACC TTG AAG GAG TGT GG
HuVH3B-For (SEQ ID NO: 26)
5′ ggc tat ggt tgc GGC TCG AGC SAG GTG CAG CTG GTG GAG TGT GG
HuVH3C-For (SEQ ID NO:27)
5′ ggc tat ggt tgc GGC TCG AGC GAG GTG CAG CTG GTG GAG WGY GG
HuVH4B-For (SEQ ID NO: 28)
5′ ggc tat ggt tgc GGC TCG AGC CAG STG CAG CTG CAG CAG TCS GG
HuVH4C-Far (SEQ ID NO: 29)
5′ ggc tat ggt tgc GGC TCG AGC CAG STG CAG CTG CAG GAG TCS GG
HuVH5B-For (SEQ ID NO: 30)
5′ ggc tat ggt tgc GGC TCG AGC GAG GTA CAG GTG GTG GAG TCT GG
HuVH6A-For (SEQ ID NO: 31)
5′ ggc tat ggt tgc GG TCG AGC CAG GTA CAG CTG GAG CAG TCA GG

Vk Forward primers (ApaL1 site is underlined)

HuVK1-For (SEQ ID NO: 32)
ggc tat ggt tgc AGT GCA CTT GAC ATC CAG WTG ACC CAG TCT CC
HuVK2-For (SEQ ID NO: 33)
ggc tat ggt tgc AGT GCA CTT GAT GTT GTG ATG AGT GAG TGT GG
HuVK3-For (SEQ ID NO: 34)
ggc tat ggt tgc AGT GGT GCA CTT GAA ATT GTG WTG ACR CAG TCT CC
HuVK4-For (SEQ ID NO: 35)
ggc tat ggt tgc AGT GCA CTT GAT ATT GTG ATG ACC CAC ACT CC
HuVK5-For (SEQ ID NO: 36)
ggc tat ggt tgc AGT GCA CTT GAA ACG ACA CTC ACG CAG TCT CC
HuVK6-For (SEQ ID NO: 37)
ggc tat ggt tgc AGT GCA CTT GAA ATT GTG CTG ACT CAG TCT CC

SECONDARY PCR PRIMERS
JH Reverse Primers (Sfi and BstEII sites are underlined)

Hu JH1/2-Sfil (SEQ ID NO: 38)
ggc tat ggt tgc ggcccctgaggcc tgatca TGA GGA GAC GGT GAC CAG GGT GCC
Ru JR3-Sfil (SEQ ID NO: 39)
ggc tat ggt tgc ggcccctgaggcc tgatca TGA AGA GAC GGT GAC CAT TGT CCC
Hu JR4/5-fil (SEQ ID NO: 40)
ggc tat ggt tgc ggcccctgaggcc tgatca TGA GGA GAC GGT GAC CAG GGT TCC
Hu JH6 -Sfil (SEQ ID NO:41)
ggc tat ggt tgc ggcccctgaggcc tgatca TGA GGA GAC GGT GAC CGT GGT CCC

Jk Reverse primers(adds Sac1 site in Fw4) (Sac1 site is underlined)

Hu Jkl/4-Sac1 (SEQ ID NO: 42)
ggc tat ggt tgc ACG TTT GAG CTC CAC CTT GGT CCC
Hu Jk2Sac1 (SEQ ID NO: 43)
ggc tat ggt tgc ACG TTT GAG CTC CAG CTT GGT CCC
Hu Jk3Sac1 (SEQ ID NO: 44)
ggc tat ggt tgc ACG TTT GAG CTC CAC TTT GGT CCC
Hu Jk5Sac1 (SEQ ID NO: 45)
ggc tat ggt tgc ACG TTT GAG CTC CAG TCG TGT CCC

JH forward primers for PCR based cloning (Xho1 site is underlined)

HuVR1b/7AFor +linker (SEQ ID NO: 46)
5′ gat ggc ggt gga tcg ggc ggt ggt gga tct gga gga ggt GGC TCG AGC CAG RTG CAG
CTG GTG CAR TCT GG
HuVR1lCFor +linker (SEQ ID NO: 47)
5′ gat ggc ggt gga tcg ggc ggt ggt gga tct gga gga ggt GGC TCG AGC SAG GTC CAG
CTG GTR CAG TCT GG
HuVH2BFor +linker (SEQ ID NO: 48)
5′ gat ggc ggt gga tcg ggc ggt ggt gga tct gga gga ggt GGC TCG AGC CAG RTC ACC
TTG AAG GAG TCT GG
HuVH3B-For +linker (SEQ ID NO: 49)
5′ gat ggc ggt gga tcg ggc ggt ggt gga tct gga gga ggt GGC TCG AGC SAG GTG CAG
CTG GTG GAG TCT GG
HuVH3C-For +linker (SEQ ID NO: 50)
5′ gat ggc ggt gga tcg ggc ggt ggt gga tct gga gga ggt GGC TCG AGC GAG GTG CAG
CTG GTG GAG WCY GG
HuVH4B-For +linker (SEQ ID NO: 51)
5′ gat ggc ggt gga tcg ggc ggt ggt gga tct gga gga ggt GGC TCG AGC CAG GTG CAG
CTA CAG CAG TGG GG
HuVH4C-For +linker (SEQ ID NO: 52)
5′ gat ggc ggt gga tcg ggc ggt ggt gga tct gga gga ggt GGC TCG AGC CAG STG CAG
CTG CAG GAG TCS GG
HuVH5B-For +linker (SEQ ID NO: 53)
5′ gat ggc ggt gga tcg ggc ggt ggt gga tct gga gga ggt GGC TCG AGC GAR GTG CAG
CTG GTG CAG TCT GG
HuVH6A-For +linker (SEQ ID NO: 54)
5′ gat ggc ggt gga tcg ggc ggt ggt gga tct gga gga ggt GGC TCG AGC GAG GTA GAG
CTG GAG GAG TGA GG

Jk Reverse primers for PCR based cloning (Sac1 site is underlined)

Hu Jk1-Linker (SEQ ID NO: 55)
5′ gct cga gcc acc tcc tcc aga tcc acc acc gcc cga tcc acc gcc atc ACG TTT GAG
CTC CAC CTT GGT CCC 3′
Hu Jk2-Linker (SEQ ID NO: 56)
5′ gct cga ggcc acc tcc tcc aga tcc acc acc gcc cga tcc acc gcc atc ACG TTT GAG
CTC GAG GTT GGT GGG 3′
Hu Jk3-Linker (SEQ ID NO: 57)
5′ gct cga gcc acc tcc tcc aga tcc acc acc gcc cga tcc acc gcc atc ACG TTT GAG
CTC GAG TTT GGT GGG 3′
Hu Jk4-Linker (SEQ ID NO: 58)
5′ gct cga gcc acc tcc tcc aga tcc aca acc gcc cga tcc acc gcc atc ACG TTT GAG
CTC CAC CTT GGT CCC 3′
Hu Jk5-Linker (SEQ ID NO: 59)
5′ gct cga gcc acc tcc tcc aga tcc acc acc gcc cga tcc acc gcc atc AGG TTT GAG
CTC CAC TCG TGT CCC 3′

### Example 3

### Construction of pWRIL-5 Phage Display Vector

An XT-H2 ScFv construct containing an anti-RAGE ScFv antibody in a VL-VH format was modified as follows to construct pWRIL-5 phage display polynucleotide vector with dummy ScFv construct (SEQ ID NO:61).

First, an Xho1 site was incorporated into the linker as described above. Second, a SacI site was incorporated into the Jk sequence as described above. Third, the 5′ Sfi1 site was changed to ApaL1 site by standard mutagenesis. In addition, 2 ApaL1 sites were removed from the pWRIL-1 vector backbone and stop codons were incorporated in 3 frames into the VL and VH gene of XT-H2 anti-RAGE ScFv sequence by standard recombinant technology to preclude the carry-over of functionally expressed but non-relevant v region sequences during cloning steps. The pWRIL-5 phage display polynucleotide vector plus dummy ScFv construct (SEQ ID NO:61) is shown in FIG. 2. The designed linker sequence of pWRIL-5, which contains a dummy ScFv contruct, is set forth in SEQ ID NO:1

pWRIL-5 was constructed by cloning this linker sequence as a Pci1/Sfi1 fragment into phage display vector pWRIL-1. The plasmid design map of pWRIL-1 is illustrated in FIG. 7 and the complete nucleotide sequence of the leading strand of pWRIL-1 is shown in SEQ ID NO:60. The promoter sequence includes nucleotides number 2361 through 2706 of SEQ ID NO:60.

### Example 4

### ScFv Library Construction

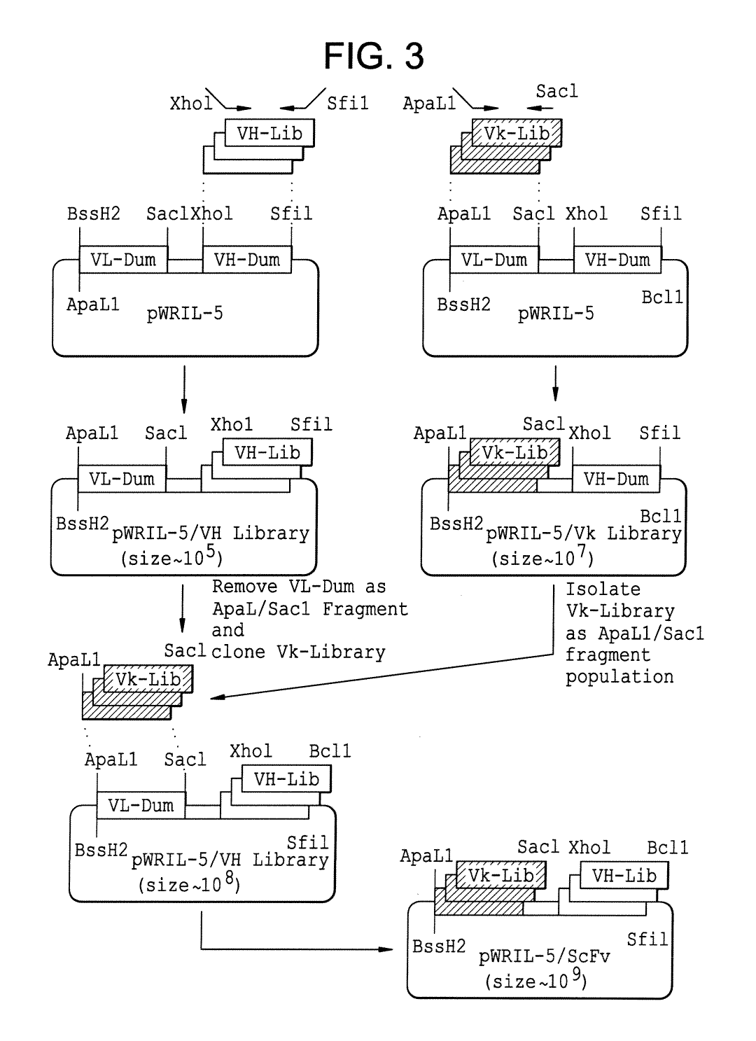

ScFv library construction is a two-step cloning procedure. The first step involves isolating and cloning the VH and VL collection separately using the amplification strategy described in Example 2. The second step involves transferring the VL collection into the VH collection, using restriction based cloning (FIG. 3).

To clone VH collection, total RNA was isolated from normal peripheral blood lymphocytes (PBLs) (about 10⁹ B cells). 1^{st} strand cDNA was synthesized using oligo dT primed cDNA synthesis. VH collection was amplified by a primary PCR using VH forward primer containing an XhoI site and an IgM specific reverse primer and a secondary PCR using the VH forward primer containing the Xho1 site and a JH reverse primer incorporating an Sfi1 site and/or BstEII site. VH gene pools were digested with Xho1/Sfi1 or Xho1/BstE11 restriction enzymes and cloned into dummy vectors to construct VH gene only collection as shown in FIG. 3.

VL collection was amplified by a primary PCR using Vk forward primer containing an ApaL1 site and Ck specific reverse primer and a secondary PCR using the Vk forward primer containing an ApaL1 site and a Jk reverse primer incorporating a linker sequence and a Sac1 site. VL gene pools were digested with ApaL1/Sac1 enzymes and cloned into dummy vector to construct Vk only collection as shown in FIG. 3.

To construct an ScFv library, the fragments containing Vk genes were isolated by digesting the Vk library with ApaL1/Sac1 enzymes. The VH only library was digested with ApaL1/Sac1 enzymes to remove VL dummy chain and the Vk fragments were cloned into the VH only library using compatible restriction sites (FIG. 3). The size of an ScFv library constructed using this method is typically 10⁹.

### Example 5

### Reformatting to Fab Phage Display/Expression Vector

An Fab expression/phage display polynucleotide vector pWRIL-6 (SEQ ID NO:62) contains compatible restrictions sites are used for reformatting ScFv to Fabs. The vector pWRIL-6 was constructed by cloning the designed linker sequence (SEQ ID NO:2) containing Ck, CH1 and ribosome binding site into vector pWRIL-1 as a Pci1/Sfi1 fragment (see Example 3). The designed linker sequence is depicted in SEQ ID NO:2.

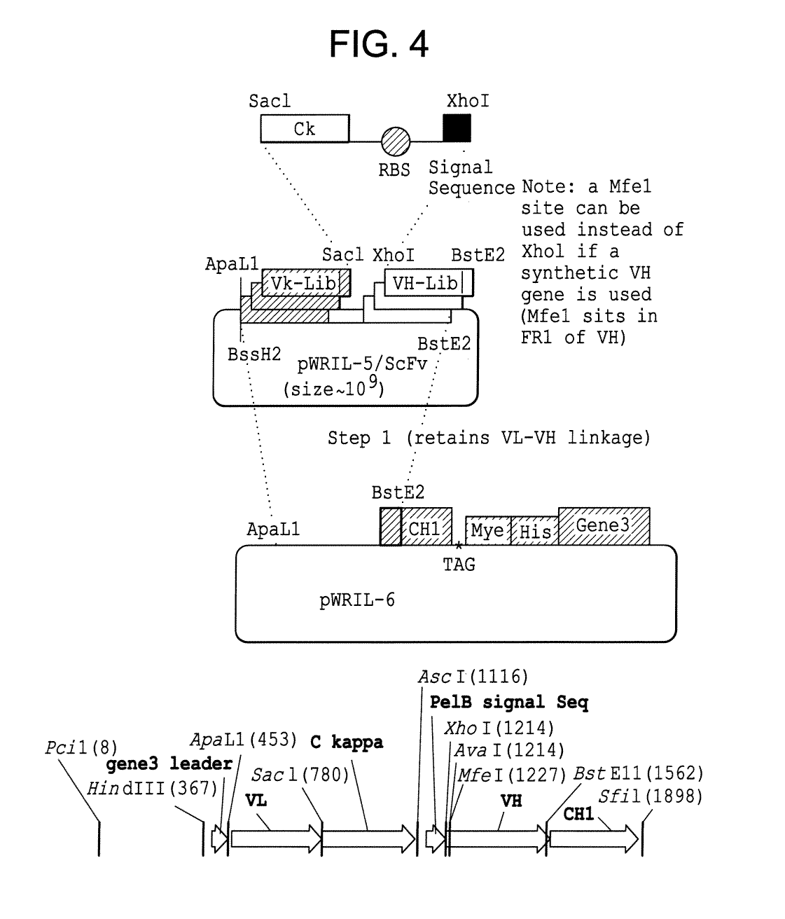

As shown in FIG. 4, an ScFv library constructed according to the present invention can be reformatted to an Fab expression system in a two-step cloning procedure independent of sequence information and retains selected VL-VH pairing during reformatting, which will allow high throughput expression and screening of Fabs. The first step involves retrieving linked VL-VH fragments from the ScFv library using ApaL1/BstEII enzyme digestion and the ApaL1/BstEII fragments are cloned into pWRIL-6 using compactible restriction sites (FIG. 4). In the second step, the linker between the VL and VH gene is replaced with a Sac1/Xho1 fragment containing Ck-rbs-PelB leader sequences (FIG. 4). If a synthetic VH gene is used, a Mfe1 site can be used instead of Xho1. The resultant pool of Fabs can be expressed and screened for potency (for example, by using BIAcore assay) or can be further selected for specific binding using phage display.

### Example 6

### Reformatting to IgG Expression System

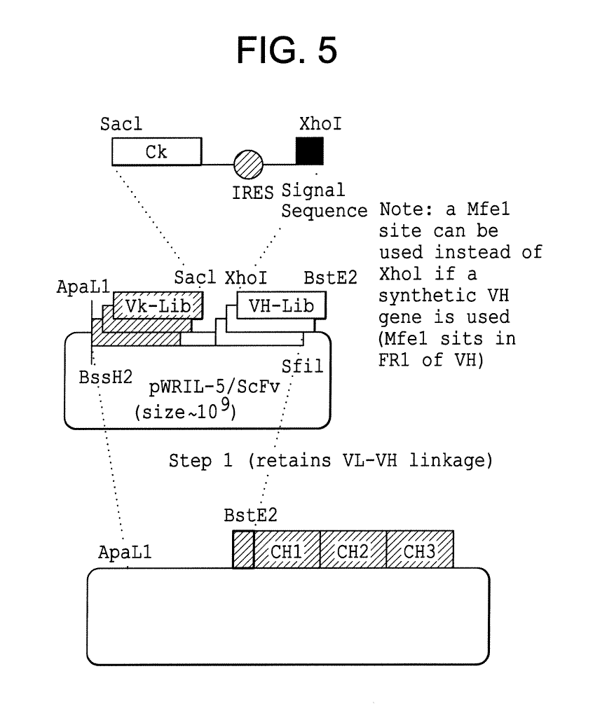

An IgG expression/phage display vector containing compatible restriction sites are used for reformatting ScFv to IgG antibodies. The IgG expression vector typically contains an Fc plus Ck-IRES signal sequence. As shown in FIG. 5, the ScFv library can be reformatted to an IgG library in a two-step cloning procedure independent of sequence information and retaining VL-VH pairing during reformatting, which allows high throughput production of IgGs by transient expression.

As discussed in Example 5, in the first step; the ScFv library is transferred into the IgG expression vectors as ApaL1/BstE11 fragments. In the second step, the linker between the VL and VH gene is replaced with a Sac1/Xho1 fragment containing Ck-IRES-signal sequences (FIG. 5). If a synthetic VH gene is used, a Mfe1 site can be used instead of Xho1. The resultant pool of IgGs can be expressed and screened for potency (for example, by using BIAcore assay) or can be further selected for specific binding using phage display.

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

All publications and patent documents cited in this application are incorporated by reference in their entirety.

What is claimed is:

1. An isolated polynucleotide comprising a nucleotide sequence, which comprises restriction enzyme recognition sites, which comprise from 5′ to 3′ an ApaLI site, a SacI site, an XhoI site and a Sfi1 site.

2. The isolated polynucleotide of claim 8, wherein the restriction enzyme recognition sites further comprise a BstEII site located 3′ to the XhoI site and 5′ to the SfiI site.

3. The isolated polynucleotide of claim 9, wherein the restriction enzyme recognition sites further comprise, from 5′ to 3′, (a) an AscI site, a PciI site and a HindIII site located 5′ to the ApaLI site, (b) an AvaI site located 3′ to the SacI site and 5′ to the XhoI site, (c) an MfeI site, an Xmal site and a Smal site located 5′ to the BstEII site, and (d) a BcII site located 3′ to the BstEII site and 5′ to the SfiI site.

4. The isolated polynucleotide of claim 3, wherein the nucleotide sequence comprises a sequence at least 95% identical to SEQ ID NO:1 or the complement thereof.

5. The isolated polynucleotide of claim 4, wherein the nucleotide sequence comprises SEQ ID NO:1 or the complement thereof.

6. The isolated polynucleotide of claim 3, wherein the nucleotide sequence comprises a sequence at least 95% identical to SEQ ID NO:61 or the complement thereof.

7. The isolated polynucleotide of claim 6, wherein the nucleotide sequence comprises SEQ ID NO:61 or the complement thereof.

8. The isolated polynucleotide of claim 2, wherein the restriction enzyme recognition sites further comprise, from 5′ to 3′, (a) a PciI site and a HindIII site located 5′ to the ApaLI site, (b) an AscI site located 3′ to the SacI site and 5′ to the XhoI site, (c) an AvaI site and a MfeI site located 3′ of the XhoI site and 5′ to the BstEII site.

9. The isolated polynucleotide of claim 8, wherein the nucleotide sequence comprises a sequence at least 95% identical to SEQ ID NO:2 or the complement thereof.

10. The isolated linker of any one of claim 9, wherein the polynucleotide sequence comprises SEQ ID NO:2 or the complement thereof.

11. The isolated polynucleotide of any one of claim 8, wherein the polynucleotide sequence comprises a sequence at least 95% identical to SEQ ID NO:62 or the complement thereof.

12. The isolated polynucleotide of any one of claim 11, wherein the polynucleotide sequence comprises SEQ ID NO:62 or the complement thereof.

13. An isolated polynucleotide comprising a VH gene, a VL gene, a XhoI restriction site positioned at the 5′ end of the VH gene, a SfiI or BstEII restriction site positioned at the 3′ end of the VH gene, an ApaL1 restriction site positioned at the 5′ end of the VL gene, and a Sac1 restriction site positioned at the 3′ end of the VL gene.

14. The isolated polynucleotide of claim 13, further comprising a polynucleotide that encodes a linker between the VH gene and the VL gene.

15. The isolated polynucleotide of claim 14, wherein the linker comprises an amino acid sequence of SEQ ID NO:3.

16. A method for constructing a single chain Fv (ScFv) polynucleotide library, the method comprising the steps of:
(a) introducing a first restriction site to the 5′ end and a second restriction site to the 3′ end of a collection of VH genes;
(b) cloning the collection of VH genes into a plurality of polynucleotide vectors using a restriction site compatible with the first restriction site and a restriction site compatible with the second restriction site;
(c) introducing a third restriction site to the 5′ end and a fourth restriction site to the 3′ end of a collection of VL genes; and
(d) cloning the collection of VL genes into the plurality of polynucleotide vectors using restriction sites compatible with the third and the fourth restriction sites;
wherein,
(i) the first, second, third and fourth restriction sites are not compatible with each other;
(ii) the first restriction site is selected from the group consisting of XhoI, SfiI, BssHI, ApaLI, MfeI, BspEI and Sall;
(iii) the second restriction site is selected from the group consisting of BstEII, SfiI, XhoI, Sall, BcII, Mlul, Smal and XbaI;
(iv) the third restriction site is selected from the group consisting of ApaLI, XhoI, Sall, BspEI, BssHII, EcoRV and SfiI; and
(v) the fourth restriction site is selected from the group consisting of SacI, SfiI, BcII, AvrII, BsiWI, BamHl, XhoI, Sail and Mlul.

17. The method of claim 16, wherein step (a) comprises synthesizing first strand cDNA from isolated total RNA and amplifying VH genes by PCR amplification using one or more primer sets comprising a forward primer comprising the first restriction site and a reverse primer comprising the second restriction site.

18. The method of claim 16, wherein step (c) comprises synthesizing first strand cDNA from isolated RNA and amplifying VL genes by PCR amplification using one or more primer sets comprising a forward primer comprising the third restriction site and a reverse primer comprising the fourth restriction site.

19. The method of claim 16, wherein each polynucleotide vector of the plurality of vectors comprises a nucleotide sequence at least 95% identical to SEQ ID NO:1.

20. The method of claim 19, wherein each polynucleotide vector of the plurality of vectors comprises a nucleotide sequence of SEQ ID NO:1.

21. The method of claim 16, wherein each polynucleotide vector of the plurality of vectors comprises a nucleotide sequence at least 95% identical to SEQ ID NO:61.

22. The method of claim 21, wherein each polynucleotide vector of the plurality of vectors comprises a nucleotide sequence of SEQ ID NO:61.

23. A single chain Fv (ScFv) polynucleotide library comprising a plurality of isolated polynucleotides, wherein each polynucleotide is a polynucleotide of claim 1.

24. A single-chain Fv (ScFv) polynucleotide library constructed using the method of claim 16.

25. A method for reformatting a single-chain Fv (ScFv) polynucleotide library into an Fab polynucleotide vector expression system, the method comprising the steps of:
(a) providing an ScFv polynucleotide library of claim 24;
(b) generating a plurality of polynucleotide fragments, each of which comprises a VH gene and a VL gene, by digesting the ScFv polynucleotide library using one or more restriction enzymes; and
(c) cloning each polynucleotide fragment of the plurality of polynucleotide fragments generated from step (b) into a Fab polynucleotide expression vector comprising compatible restriction sites.

26. The method of claim 25, wherein each of the plurality of fragments further comprises a polynucleotide that encodes a linker between the VH and the VL gene.

27. The method of claim 26, wherein the linker comprises an amino acid sequence of SEQ ID NO:3.

28. The method of claim 26, wherein the method further comprises a step of replacing at least a portion of the polynucleotide that encodes a linker with a nucleotide sequence comprising a Ck sequence, a ribosome binding site (rbs) and a signal peptide sequence.

29. The method of claim 28, wherein the signal peptide sequence comprises a PelB leader sequence.

30. The method of claim 25, wherein each Fab polynucleotide expression vector of the plurality of Fab polynucleotide expression vectors comprises a nucleotide sequence at least 95% identical to SEQ ID NO:2.

31. The method of claim 30, wherein each Fab polynucleotide expression vector of the plurality of Fab polynucleotide expression vectors comprises a nucleotide sequence of SEQ ID NO:2.

32. The method of claim 25, wherein each Fab polynucleotide expression vector of the plurality of Fab polynucleotide expression vectors comprises a nucleotide sequence at least 95% identical to SEQ ID NO:62.

33. The method of claim 32, wherein each Fab polynucleotide expression vector of the plurality of Fab polynucleotide expression vectors comprises a nucleotide sequence of SEQ ID NO:62.

34. A polynucleotide library constructed according of claim 25, wherein the polynucleotide library comprises a plurality of ScFv polynucleotides in a plurality of Fab polynucleotide expression vectors.

35. A method for reformatting a single-chain Fv (ScFv) polynucleotide library into an IgG expression system, the method comprising the steps of:
(a) providing an ScFv polynucleotide library of claim 24;
(b) generating a plurality of polynucleotide fragments, each of which comprises a VH gene and a VL gene, by digesting the ScFv library using one or more restriction enzymes; and
(c) cloning the each polynucleotide fragment of the plurality of polynucleotide fragments generated from step (b) to an IgG polynucleotide expression vectors comprising compatible restriction sites.

36. The method of claim 35, wherein each polynucleotide of the plurality of polynucleotide fragments further comprises a polynucleotide that encodes a linker between the VH and the VL gene.

37. The method of claim 36, wherein the linker comprises an amino acid sequence of SEQ ID NO:3.

38. The method of claim 36, wherein the method further comprises a step of replacing at least a portion of the polynucleotide that encodes a linker with a sequence comprising a Ck sequence, an internal ribosome entry site (IRES) and a signal peptide sequence.

39. The method of claim 35, wherein the IgG polynucleotide expression vector comprises a nucleotide sequence at least 95% identical to SEQ ID NO:2.

40. The method of claim 39, wherein the IgG polynucleotide expression vector comprises a nucleotide sequence of SEQ ID NO:2.

41. A polynucleotide library constructed according to claim 35, wherein the polynucleotide library comprises a plurality of ScFv polynucleotides in a plurality of IgG polynucleotide expression vectors.

42. A method for producing a VH polypeptide and a VL polypeptide comprising:
(a) digesting the polynucleotide library of claim 41 with a second restriction enzyme that recognizes the second restriction site and a third restriction enzyme that recognizes the third restriction site, wherein each polynucleotide of the polynucleotide library comprises a polynucleotide that encodes a linker located between the second restriction site and the third restriction site such that the polynucleotide that encodes the linker is released from the Fab or IgG expression vector thereby creating (i) a linear vector polynucleotide fragment comprising a polynucleotide that encodes the VH polypeptide and a polynucleotide that encodes the VH polypeptide (“linear vector polynucleotide”) and (ii) a linear polynucleotide fragment that encodes the linker (“linear linker polynucleotide”);
(b) isolating the linear vector polynucleotide;
(c) ligating a polynucleotide comprising a regulatory sequence (“expression cassette”) to the linear vector polynucleotide at the second restriction site and the third restriction site to form a polynucleotide that separately encodes a VH polypeptide and a VL polypeptide (“VH and VL expression vector polynucleotide”); and
(d) inducing expression of the VH polypeptide and the VL polypeptide from the VH and VL expression vector polynucleotide,
wherein a VH polypeptide and a VL polypeptide are produced.

43. The method according to claim 42, wherein the VH polypeptide is fused to a heavy chain constant regions 1 and the VL polypeptide is fused to a kappa chain constant region or a lambda chain constant region.

44. The method according to claim 42, wherein the VH polypeptide is fused to heavy chain constant regions 1, 2 and 3, and the VL polypeptide is fused to a kappa chain constant region or a lambda chain constant region.
